SEQUENCE LISTING (2 sequences)

SEQ ID NO: 1 - RNA, 16 nt, Artificial Sequence
  Synthetic Construct
atgcaactacaatgca

SEQ ID NO: 2 - RNA, 21 nt, Artificial Sequence
  Synthetic Construct
tgcaatgcaactacaatgcac

(19) United States
(12) Patent Application Publication
(10) Pub. No.: US 2022/0195428 A1
(43) Pub. Date: Jun. 23, 2022

(54) OLIGONUCLEOTIDES AND METHODS FOR THE TREATMENT OF AGE-RELATED MACULAR DEGENERATION

(71) Applicants: The Genera Hospital Corporation, Boston, MA (US); The Schepens Eye Research Institute, Inc., Boston, MA (US)

(72) Inventors: Anders M. NAAR, Berkeley, CA (US); Patricia D'AMORE, Newton, MA (US)

(21) Appl. No.: 17/310,235

(22) PCT Filed: Jan. 29, 2020
(86) PCT No.: PCT/US20/15638

§ 371 Date: Jul. 27, 2021

Related U.S. Application Data
(60) Provisional application No. 62/798,048, filed on Jan. 29, 2019.

(51) Int. Cl. C12N 15/113; C12N 15/86; A61P 27/02; A61K 31/7088

(52) U.S. Cl. CPC C12N 15/113; C12N 15/86; A61P 27/02; A61K 31/7088; C12N 2310/315; C12N 2310/113; C12N 2750/14043; C12N 2750/14023; C12N 2310/3231

(57) ABSTRACT

Disclosed are oligonucleotides, compositions, and methods that may be useful in the treatment of age-related macular degeneration (AMD). The treatment of age-regulated macular degeneration (AMD) may involve inhibiting an miR-33 target nucleic acid. For example, inhibition of an miR-33 target nucleic acid may be achieved using antisense oligonucleotides targeting an miR-33 target nucleic acid, interfering oligonucleotides targeting an miR-33 target nucleic acid, or recombinant AAV particles including a vector encoding an antisense oligonucleotide or interfering oligonucleotide targeting an miR-33 target nucleic acid.

### CROSS-REFERENCE TO RELATED APPLICATIONS

This application claims the benefit of U.S. Provisional Application No. 62/798,048, filed Jan. 29, 2019, which is incorporated herein by reference in its entirety.

### STATEMENT REGARDING FEDERALLY SPONSORED RESEARCH OR DEVELOPMENT

The invention was made with government support under Grant Nos. P30 EY003790 and RO1 HL111932, awarded by the National Institutes of Health. The government has certain rights in the invention.

### REFERENCE TO AN ELECTRONICALLY SUBMITTED SEQUENCE LISTING

The instant application contains a Sequence Listing which has been submitted electronically in ASCII format and is hereby incorporated by reference in its entirety. Said ASCII copy, created on Jan. 28, 2020 is named 51319-003W02_Sequence_Listing_01.28.20_ST25 and is 735 bytes in size.

### FIELD OF THE INVENTION

The invention relates to oligonucleotides, rAAV particles, their pharmaceutical compositions, and methods of their use for the treatment of, e.g., macular degeneration.

### BACKGROUND

MicroRNAs (miRNAs) are small (approximately 21-24 nucleotides in length, these are also known as “mature” miRNA), non-coding RNA molecules encoded in the genomes of plants and animals. These highly conserved, endogenously expressed RNAs are believed to regulate the expression of genes by binding to the 3′-untranslated regions (3′-UTR) of specific mRNAs. MiRNAs may act as key regulators of cellular processes such as cell proliferation, cell death (apoptosis), metabolism, and cell differentiation. On a larger scale, miRNA expression has been implicated in early development, brain development, disease progression (such as cancers and viral infections). There is speculation that in higher eukaryotes, the role of miRNAs in regulating gene expression could be as important as that of transcription factors. Numerous different miRNAs have been identified. Mature miRNAs appear to originate from long endogenous primary miRNA transcripts (also known as pri-miRNAs, pri-mirs, pri-miRs or pri-pre-miRNAs) that are often hundreds of nucleotides in length.

In mammals, only a few miRNAs have been assigned any function, although they are predicted to regulate a large percentage of genes, with estimates based on bioinformatic target prediction ranging as high as 30%.

Age-related macular degeneration (AMD) is a progressive chronic disease of the central retina with significant consequences for visual acuity. Late forms of the disease are the leading cause of vision loss in industrialized countries. For the Caucasian population 40 years of age, the prevalence of early AMD is estimated at about 6.8% and advanced AMD at about 1.5%. The prevalence of late AMD increases dramatically with age rising to about 11.8% after 80 years of age. Two types of AMD exist, non-exudative (dry) and exudative (wet) AMD. The more common dry AMD involves atrophic and hypertrophic changes in the retinal pigment epithelium (RPE) underlying the central retina (macula) as well as deposits (drusen) on the RPE. Advanced dry AMD can result in significant retinal damage, including geographic atrophy (GA), with irreversible vision loss. Moreover, patients with dry AMD can progress to the wet form, in which abnormal blood vessels called choroidal neovascular membranes (CNVMs) develop under the retina, leak fluid and blood, and ultimately cause a blinding disciform scar in and under the retina.

There is a need for new therapeutic approaches to the treatment of age-related macular degeneration.

### SUMMARY OF THE INVENTION

In one aspect, the invention provides an oligonucleotide including a total of 7 to 50 interlinked nucleotides and having a nucleobase sequence including at least 6 contiguous nucleobases complementary to an equal-length portion within an miR-33 target nucleic acid. In some embodiments, at least one nucleotide in the oligonucleotide is a bridged nucleic acid.

In some embodiments, the oligonucleotide is an antisense oligonucleotide. In some embodiments, the oligonucleotide is a single-stranded oligonucleotide. In some embodiments, the oligonucleotide is a unimer, and where each of the nucleotides is independently a bridged nucleic acid. In some embodiments, the bridged nucleic acid is a locked nucleic acid or ethylene bridged nucleic acid. In some embodiments, the bridged nucleic acid is a locked nucleic acid. In some embodiments, the oligonucleotide includes a total of 7 to 30 nucleotides (e.g., 14 to 23 nucleotides). In some embodiments, the miR-33 target nucleic acid is pri-miR-33a, pre-miR-33a, or miR-33a. In some embodiments, the miR-33 target nucleic acid is pri-miR-33b, pre-miR-33b, or miR-33b. In some embodiments, the nucleobase sequence is 5′-ATGCAACTACAATGCA-3′ (SEQ ID NO: 1). In some embodiments, the nucleobase sequence is 5′-TGCAATGCAACTACAATGCAC-3′ (SEQ ID NO: 2).

In another aspect, the invention provides a recombinant adeno-associated viral (rAAV) particle including a nucleic acid vector that includes a heterologous nucleic acid region including a sequence that encodes an interfering RNA including a region complementary to an miR-33 target nucleic acid.

In some embodiments, the miR-33 target nucleic acid is pri-miR-33a, pre-miR-33a, or miR-33a. In some embodiments, the miR-33 target nucleic acid is pri-miR-33b, pre-miR-33b, or miR-33b. In some embodiments, the interfering RNA is shRNA or siRNA. In some embodiments, the sequence is operably linked to a promoter. In some embodiments, the promoter is capable of expressing the interfering RNA in a subject's eye. In some embodiments, the promoter is a hybrid chicken β-actin (CBA) promoter or an RNA polymerase III promoter. In some embodiments, the vector includes AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats. In some embodiments, the particle includes an AAV1, AAV2, AAV3, AAV4, AAV5, AAV6, AAV7, AAV8, AAVrh8, AAVrh8R, AAV9, AAV10, AAVrh10, AAV11, AAV12, a tyrosine capsid mutant, a heparin binding capsid mutant, an AAV2R471A capsid, an AAVAAV2/2-7m8 capsid, an AAV DJ capsid, AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, or rAAV2/HBoV1 capsid.

In yet another aspect, the invention provides a pharmaceutical composition including a pharmaceutically acceptable excipient and the oligonucleotide described herein or the rAAV particle described herein.

In still another aspect, the invention provides a method of treating age-related macular degeneration in a subject in need thereof.

In some embodiments, the method includes administering to the subject a therapeutically effective amount of the oligonucleotide of described herein, the rAAV particle described herein, or the pharmaceutical composition described herein. In some embodiments, the method includes administering to the subject a therapeutically effective amount of an miR-33 inhibitor (e.g., an antisense oligonucleotide, shRNA, siRNA, or an rAAV particle including a nucleic acid vector that includes a heterologous nucleic acid region including a sequence that encodes the miR-33 inhibiting antisense oligonucleotide, shRNA, or siRNA). In some embodiments, the method includes administering to the subject a therapeutically effective amount of an oligonucleotide including a total of 7 to 50 interlinked nucleotides and having a nucleobase sequence including at least 6 contiguous nucleobases complementary to an equal-length portion within an miR-33 target nucleic acid. In some embodiments, the method includes administering to the subject a therapeutically effective amount of a recombinant adeno-associated viral (rAAV) particles including a nucleic acid vector that includes a heterologous nucleic acid region including a sequence that encodes the oligonucleotide described herein (e.g., shRNA or siRNA).

In some embodiments, the method includes administering a therapeutically effective amount of the oligonucleotide. In some embodiments, the oligonucleotide is a single-stranded oligonucleotide. In some embodiments, the oligonucleotide is an antisense oligonucleotide. In some embodiments, the oligonucleotide includes at least one modified sugar nucleoside. In some embodiments, at least 50% (e.g., at least 60%, at least 70%, at least 80%, at least 90%, or all) of the nucleosides in the oligonucleotide include the modified sugar nucleoside. In some embodiments, the modified sugar nucleoside is a 2′-modified sugar nucleoside (e.g., a 2′-modified sugar nucleoside including a 2′-modification independently selected from the group consisting of 2′-fluoro, 2′-methoxy, and 2′-methoxyethoxy). In some embodiments, the modified sugar nucleoside is a bridged nucleic acid.

In some embodiments, the oligonucleotide is a gapmer including a 5′-wing, a 3′-wing, and a gap; where each of the 5′-wing and the 3′-wing includes a total of 1 to 5 nucleotides, each of which is independently a bridged nucleic acid, and each nucleotide in the gap a deoxyribonucleotide. In some embodiments, the bridged nucleic acid is a locked nucleic acid or ethylene bridged nucleic acid. In some embodiments, the bridged nucleic acid is a locked nucleic acid. In some embodiments, at least one internucleoside linkage in the oligonucleotide is a phosphorothioate diester. In some embodiments, at least 50% (e.g., at least 60%, at least 70%, at least 80%, at least 90%, or all) of the internucleoside linkages in the oligonucleotide are phosphorothioate diesters. In some embodiments, the nucleobase sequence is 5′-ATGCAACTACAATGCA-3′ (SEQ ID NO: 1). In some embodiments, the nucleobase sequence is 5′-TGCAATGCAACTACAATGCAC-3′ (SEQ ID NO: 2). In some embodiments, the oligonucleotide includes a total of 7 to 30 nucleotides (e.g., 14 to 23 nucleotides).

In some embodiments, the method includes administering the oligonucleotide as a guide strand in an siRNA.

In some embodiments, the method includes administering the rAAV particle (e.g., a rAAV particle described herein).

In some embodiments, the miR-33 target nucleic acid is pri-miR-33a, pre-miR-33a, or miR-33a. In some embodiments, the miR-33 target nucleic acid is pri-miR-33b, pre-miR-33b, or miR-33b.

In some embodiments, the route of administration is an intraocular injection, intravitreal injection, subretinal injection, topical application, implantation, intraperitoneal injection, intramuscular injection, subcutaneous injection, or intravenous injection.

The invention is also described by the following enumerated items.

1. An oligonucleotide including a total of 7 to 50 interlinked nucleotides and having a nucleobase sequence including at least one bridged nucleic acid and at least 6 contiguous nucleobases complementary to an equal-length portion within an miR-33 target nucleic acid.

2. The oligonucleotide of item 1, where the oligonucleotide is an antisense oligonucleotide.

3. The oligonucleotide of item 1 or 2, where the oligonucleotide is a single-stranded oligonucleotide.

4. The oligonucleotide of any one of items 1 to 3, where the oligonucleotide is a unimer, and where each of the nucleotides is independently a bridged nucleic acid.

5. The oligonucleotide of any one of items 1 to 4, where the bridged nucleic acid is a locked nucleic acid or ethylene bridged nucleic acid.

6. The oligonucleotide of item 5, where the bridged nucleic acid is a locked nucleic acid.

7. The oligonucleotide of any one of items 1 to 6, where the oligonucleotide includes a total of 7 to 30 nucleotides.

8. The oligonucleotide of any one of items 1 to 6, where the oligonucleotide includes a total of 14 to 23 nucleotides.

9. The oligonucleotide of any one of items 1 to 8, where the miR-33 target nucleic acid is pri-miR-33a, pre-miR-33a, or miR-33a.

10. The oligonucleotide of any one of items 1 to 8, where the miR-33 target nucleic acid is pri-miR-33b, pre-miR-33b, or miR-33b.

11. The oligonucleotide of any one of items 1 to 6, where the nucleobase sequence is 5′-ATGCAACTACAATGCA-3′ (SEQ ID NO: 1).

12. The oligonucleotide of any one of items 1 to 5, wherein the nucleobase sequence is 5′-TGCAATGCAACTACAATGCAC-3′ (SEQ ID NO: 2).

13. A recombinant adeno-associated viral (rAAV) particle including a nucleic acid vector that includes a heterologous nucleic acid region including a sequence that encodes an interfering RNA including a region complementary to an miR-33 target nucleic acid.

14. The rAAV particle of item 13 where the miR-33 target nucleic acid is pri-miR-33a, pre-miR-33a, or miR-33a.

15. The rAAV particle of item 13, where the miR-33 target nucleic acid is pri-miR-33b, pre-miR-33b, or miR-33b.

16. The rAAV particle of any one of items 13 to 15, where the interfering RNA is shRNA or siRNA.

17. The rAAV particle of any one of items 13 to 16, where the sequence is operably linked to a promoter.

18. The rAAV particle of item 17, where the promoter is capable of expressing the interfering RNA in a subject's eye.

19. The rAAV particle of item 17 or 18, where the promoter is a hybrid chicken β-actin (CBA) promoter or an RNA polymerase III promoter.

20. The rAAV particle of any one of items 13 to 19, where the vector includes AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats.

21. The rAAV particle of any one of items 13 to 20, where the particle includes an AAV1, AAV2, AAV3, AAV4, AAV5, AAV6, AAV7, AAV8, AAVrh8, AAVrh8R, AAV9, AAV10, AAVrh10, AAV11, AAV12, a tyrosine capsid mutant, a heparin binding capsid mutant, an AAV2R471A capsid, an AAVAAV2/2-7m8 capsid, an AAV DJ capsid, AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, or rAAV2/HBoV1 capsid.

22. A pharmaceutical composition including a pharmaceutically acceptable excipient and the oligonucleotide of any one of items 1 to 11 or the rAAV particle of any one of items 12 to 21.

23. A method of treating age-related macular degeneration in a subject in need thereof, the method including administering to the subject a therapeutically effective amount of the oligonucleotide of any one of items 1 to 11, the rAAV particle of any one of items 12 to 21, or the pharmaceutical composition of item 22.

24. A method of treating age-related macular degeneration in a subject in need thereof, the method including administering to the subject a therapeutically effective amount of an miR-33 inhibitor.

25. The method of item 24, where the miR-33 inhibitor is an antisense oligonucleotide, shRNA, siRNA, or an rAAV particle including a nucleic acid vector that includes a heterologous nucleic acid region including a sequence that encodes the miR-33 inhibiting antisense oligonucleotide, shRNA, or siRNA.

26. A method of treating age-related macular degeneration in a subject in need thereof, the method including administering to the subject a therapeutically effective amount of:
(i) an oligonucleotide including a total of 7 to 50 interlinked nucleotides and having a nucleobase sequence including at least 6 contiguous nucleobases complementary to an equal-length portion within an miR-33 target nucleic acid;
or
(ii) a recombinant adeno-associated viral (rAAV) particles including a nucleic acid vector that includes a heterologous nucleic acid region including a sequence that encodes the oligonucleotide.

27. The method of item 26, where the method includes administering a therapeutically effective amount of the oligonucleotide.

28. The method of item 27, where the oligonucleotide is a single-stranded oligonucleotide.

29. The method of item 27 or 28, where the oligonucleotide is an antisense oligonucleotide.

30. The method of item 28 or 29, where the oligonucleotide includes at least one modified sugar nucleoside.

31. The method of item 30, where at least 50% of the nucleosides in the oligonucleotide include the modified sugar nucleoside.

32. The method of item 30 or 31, where all nucleosides in the oligonucleotide include the modified sugar nucleoside.

33. The method of any one of items 30 to 32, where the modified sugar nucleoside is a 2′-modified sugar nucleoside.

34. The method of item 33, where the 2′-modified sugar nucleoside includes a 2′-modification independently selected from the group consisting of 2′-fluoro, 2′-methoxy, and 2′-methoxyethoxy.

35. The method of any one of items 30 to 32, where the modified sugar nucleoside is a bridged nucleic acid.

36. The method of any one of items 27 to 32, where the oligonucleotide is a gapmer including a 5′-wing, a 3′-wing, and a gap; where each of the 5′-wing and the 3′-wing includes a total of 1 to 5 nucleotides, each of which is independently a bridged nucleic acid, and each nucleotide in the gap a deoxyribonucleotide.

37. The method of item 35 or 36, where the bridged nucleic acid is a locked nucleic acid or ethylene bridged nucleic acid.

38. The method of item 37, where the bridged nucleic acid is a locked nucleic acid.

39. The method of any one of items 27 to 38, where at least one internucleoside linkage in the oligonucleotide is a phosphorothioate diester.

40. The method of item 39, where at least 50% of internucleoside linkages in the oligonucleotide are phosphorothioate diesters.

41. The method of item 40, where all internucleoside linkages in the oligonucleotide are phosphorothioate diesters.

42. The method of any one of items 27 to 41, where the nucleobase sequence is 5′-ATGCAACTACAATGCA-3′ (SEQ ID NO: 1).

43. The method of any one of items 27 to 41, where the nucleobase sequence is 5′-TGCAATGCAACTACAATGCAC-3′ (SEQ ID NO: 2).

44. The method of any one of items 28 to 41, where the oligonucleotide includes a total of 7 to 30 nucleotides.

45. The method of any one of items 28 to 41, where the oligonucleotide includes a total of 14 to 23 nucleotides.

46. The method of item 27, where the method includes administering the oligonucleotide as a guide strand in an siRNA.

47. The method of item 26, where the method includes administering the rAAV particle.

48. The method of item 47, where the rAAV particle is that of any one of items 13 to 21.

49. The method of any one of items 26 to 48, where the miR-33 target nucleic acid is pri-miR-33a, pre-miR-33a, or miR-33a.

50. The method of any one of items 26 to 48, where the miR-33 target nucleic acid is pri-miR-33b, pre-miR-33b, or miR-33b.

51. The method of any one of items 23 to 50, where the route of administration is an intraocular injection, intravitreal injection, subretinal injection, topical application, implantation, intraperitoneal injection, intramuscular injection, subcutaneous injection, or intravenous injection.

### Definitions

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contain several shorter regions of self-complementarity (designated A, A′, B, B′, C, C′ and D regions), allowing intrastrand base-pairing to occur within this portion of the ITR

The term “about,” as used herein, represents a value that is in the range of ±10% of the value that follows the term “about.”

“Antisense” to a target nucleic acid when, written in the 5′ to 3′ direction, it includes the reverse complement of the corresponding region of the target nucleic acid. Such antisense compounds are known as “antisense oligonucleotides,” which include, without limitation, oligonucleotides, oligonucleosides, or oligonucleotide analogs. In general, an antisense oligonucleotide includes a backbone of linked monomeric subunits, where each linked monomeric subunit is a nucleotide. The internucleoside linkages, the nucleoside sugars, and the nucleobases may be independently modified giving rise to antisense oligonucleotides motifs, e.g., hemimers, gapmers, alternating, uniformly modified, and positionally modified. The antisense oligonucleotides described herein include a total of 7 to 50 contiguous nucleotides. Non-limiting examples of antisense oligonucleotides include those having a total of 7, 8, 9, 10, 11, 12, 13, 14, 15, 16, 17, 18, 19, 20, 21, 22, 23, 24, 25, 26, 27, 28, 29, or 30 nucleotides.

“Chicken β-actin (CBA) promoter” refers to a polynucleotide sequence derived from a chicken β-actin gene (e.g., Gallus gallus beta actin, represented by GenBank Entrez Gene ID 396526). As used herein, “chicken β-actin promoter” may refer to a promoter containing a cytomegalovirus (CMV) early enhancer element, the promoter and first exon and intron of the chicken β-actin gene, and the splice acceptor of the rabbit beta-globin gene, such as the sequences described in Miyazaki, J., et al. (1989) Gene 79(2):269-77. As used herein, the term “CAG promoter” may be used interchangeably. As used herein, the term “CMV early enhancer/chicken beta actin (CAG) promoter” may be used interchangeably.

The term “complementary,” as used herein, refers to the capacity for hybridization of two nucleobases. Conversely, a position is considered “non-complementary” when nucleobases are not capable of hybridizing according to Watson-Crick pairing, Hoogsteen pairing, or reverse Hoogsteen pairing. An antisense compound and a target nucleic acid are “fully complementary” to each other when each nucleobase of the antisense compound is complementary to an equal number of nucleobases at corresponding positions in the target nucleic acid.

The term “gapmer,” as used herein, refers to an oligonucleotide strand including a 5′-wing, 3′-wing, and a gap. Each of the 3′-wing and 5′-wing is typically modified to include one or more affinity enhancing nucleosides (e.g., bridged nucleic acids). All internucleoside linkages in a gapmer may be, e.g., phosphate diesters, phosphorothioate diesters, or a combination thereof.

The term “heterologous,” as used herein, means derived from a genotypically distinct entity from that of the rest of the entity to which it is compared or into which it is introduced or incorporated. For example, a nucleic acid introduced by genetic engineering techniques into a different cell type is a heterologous nucleic acid (and, when expressed, can encode a heterologous polypeptide). Similarly, a cellular sequence (e.g., a gene or portion thereof) that is incorporated into a viral vector is a heterologous nucleotide sequence with respect to the vector.

An “inverted terminal repeat” or “ITR” sequence is a term well understood in the art and refers to relatively short sequences found at the termini of viral genomes which are in opposite orientation. In the rAAV particles described herein, ITR sequences are typically AAV inverted terminal repeat (ITR) sequences.

The term “miR-33 or a precursor thereof,” as used herein, refers to miR-33a, pre-miR-33a, pri-miR-33a, miR-33b, pre-miR-33b, pri-miR-33b, or a primary RNA transcript from which miR-33a and miR-33b are eventually derived.

The term “miR-33 target nucleic acid,” as used herein, refers to pri-miR-33a, pre-miR-33a, miR-33a, pri-miR-33b, pre-miR-33b, or miR-33b. In the context of the present disclosure, pri-miR-33a and pri-miR-33b are primary miRNAs, pre-miR-33a and pre-miR-33b are pre-miRNAs, and miR-33a and miR-33b are mature miRNAs. “Mature miR-33a” and “miR-33a” may be used interchangeably herein. “mature miR-33b” and “miR-33b” may be used interchangeably herein. MiR-33a and miR-33b differ by 2 of 19 nucleotides in their mature form but are identical in the seed sequence which dictates binding to the 3′UTR of genes. A human pre-miR-33a is described in NCBI Reference Sequence: NR_029507.1. A human pre-miR-33b is described in NCBI Reference Sequence: NR_030361.1. A human miR-33a is described in NCBI GenBank: AJ421755.1. A human miR-33b is described in NCBI GenBank: AJ550398.1.

The term “nucleoside,” as used herein, represents sugar-nucleobase compounds and groups known in the art, as well as modified or unmodified 2′-deoxyribofuranose-nucleobase compounds and groups known in the art. The sugar may be, e.g., ribofuranose, 2′-deoxyribofuranose, or bridged furanose (e.g., a bridged furanose that is found in bridged nucleic acids). The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a locking modification are known in the art as bridged nucleic acids, e.g., locked nucleic acids (LNA; the locking modification is a 4′-CH₂O-2′ bridge), ethylene-bridged nucleic acids (ENA; the locking modification is a 4′-CH₂CH₂O-2′ bridge), and cEt nucleic acids (the locking modification is an (R)-4′-CH(CH₃)—O-2′ or (S)-4′-CH(CH₃)—O-2′ bridge). The bridged nucleic acids are typically used as affinity enhancing nucleosides.

The term “nucleotide,” as used herein, represents a nucleoside bonded to an internucleoside linkage.

The term “oligonucleotide,” as used herein, represents a structure containing 10 or more contiguous nucleosides covalently bound together by internucleoside linkages. An oligonucleotide includes a 5′ end and a 3′ end. The 3′ and 5′ ends may be substituted using groups known in the art. Oligonucleotides can be in double- or single-stranded form. Double-stranded oligonucleotide molecules can optionally include one or more single-stranded segments (e.g., overhangs).

The term “pharmaceutical composition,” as used herein, represents a composition formulated with an oligonucleotide disclosed herein and one or more pharmaceutically acceptable excipients, and manufactured or sold as part of a therapeutic regimen for the treatment of disease in a mammal.

The term “pharmaceutically acceptable excipient,” as used herein, refers to any ingredient other than the oligonucleotide described herein (e.g., a vehicle capable of suspending or dissolving the active compound) and having the properties of being substantially non-toxic and substantially non-inflammatory in a patient. Excipients may include, e.g., antioxidants, disintegrants, dyes (colors), emollients, emulsifiers, fillers (diluents), flavors, fragrances, preservatives, printing inks, sorbents, suspending or dispersing agents, sweeteners, liquid solvents, and buffering agents.

An “rAAV virus” or “rAAV viral particle” refers to a viral particle composed of at least one AAV capsid protein and an encapsidated rAAV vector genome.

The term “recombinant AAV vector (rAAV vector),” as used herein, refers to a polynucleotide vector comprising one or more heterologous sequences (i.e., nucleic acid sequence not of AAV origin) that are flanked by at least one, preferably two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and, in embodiments, encapsidated in a viral particle, particularly an AAV particle. A rAAV vector can be packaged into an AAV virus capsid to generate a “recombinant adeno-associated viral particle (rAAV particle)”. AAV helper functions (i.e., functions that allow AAV to be replicated and packaged by a host cell) can be provided in any of a number of forms, including, but not limited to, helper virus or helper virus genes which aid in AAV replication and packaging. Other AAV helper functions are known in the art.

As used herein “RNA interference (RNAi)” is a biological process in which RNA molecules cause degradation of targeted small non-coding RNA. Examples of RNAi include small inhibitory RNA (siRNA) and small hairpin RNA (shRNA).

The term “siRNA,” as used herein, refers to a double-stranded oligonucleotide including an antisense sequence that is complementary to a target RNA and a sense sequence that is the reverse complement of the antisense sequence. An antisense sequence is typically referred to as a guide strand, and a sense sequence is typically referred to as a passenger strand.

As used herein, the term “small non-coding RNA” is used to encompass, without limitation, a polynucleotide molecule ranging from about 17 to about 450 nucleosides in length, which can be endogenously transcribed or produced exogenously (chemically or synthetically), but is not translated into a protein. As is known in the art, primary miRNAs (also known as pri-pre-miRNAs, pri-miRs, and pri-miRNAs) range from around 70 nucleosides to about 450 nucleosides in length and often take the form of a hairpin structure. The primary miRNA is believed to be processed by Drosha to yield a pre-miRNA (also known as pre-miRs and foldback miRNA precursors), which ranges from around 50 nucleosides to around 110 nucleosides in length. It is believed that the pre-miRNA is in turn processed by Dicer to yield a miRNA (also known as microRNA, miR, and mature miRNA), which ranges from 19 to 24 nucleosides in length. Small non-coding RNAs may include isolated single-, double-, or multiple-stranded molecules, any of which may include regions of intrastrand nucleobase complementarity, said regions capable of folding and forming a molecule with fully or partially double-stranded or multiple-stranded character based on regions of perfect or imperfect complementarity.

As used herein, a “small hairpin RNA” or “short hairpin RNA” (shRNA) is a RNA molecule that makes a tight hairpin turn that can be used to silence target gene expression; for example, by RNA interference.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

A “therapeutically effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “unimer,” as used herein, refers to an oligonucleotide strand, whose pattern of structural features characterizing each individual nucleotide unit is such that all nucleotide units within the strand share at least one common structural feature, e.g., a common internucleoside linkage modification or a common nucleoside sugar modification.

The term “vector,” as used herein, refers to a recombinant plasmid or virus that includes a nucleic acid to be delivered into a host cell, either in vitro or in vivo.

### BRIEF DESCRIPTION OF THE DRAWINGS

FIG. 1A-FIG. 1E show miR-33 modulated ABCA1 expression and cholesterol efflux in RPE cells. FIG. 1A shows the expression of ABCA1 as analyzed by quantitative RT-PCR in RPE cells isolated from C57BL/6J mice (n≥6) at indicated time points. FIG. 1B is a western blot showing the expression of ABCA1 and SIRT6 in ARPE-19 cells 72 hours after transfection with precursor miR control, miR-33a, or miR-33b. FIG. 1C shows western blotting demonstrating ABCA1 and SIRT6 levels in ARPE-19 cells 72 hours post-transfection with control, anti-miR-33a, anti-miR-33b, or anti-miR-33a/b ASO. FIG. 1D shows TopFluor® cholesterol efflux as measured in ARPE-19 cells transfected with precursor control miR, miR-33a or miR-33b. FIG. 1E shows TopFluor® cholesterol efflux was assessed in ARPE-19 cells ˜60 hours after transfection with scrambled control, anti-miR-33a, anti-miR-33b, or -miR-33a/b ASO. pC: precursor scrambled control miR, aC: anti-miR control. All error bars represent ±SEM. FIG. 1A shows the statistical significance between groups (n≥6) were calculated by one-way analysis of variance, followed by Dunnett's multiple comparisons test. (FIG. 1B—FIG. 1E) Blot from three independent experiments were represented and the expression levels were normalized to vinculin loading control and statistical significance between groups was calculated by unpaired t test. (FIG. 1D-FIG. 1E) Each experiment was performed in quadruplicates and repeated 3 times and statistical significance between groups was calculated by unpaired t test. *P<0.05, **P<0.01, ***P<0.001

FIG. 2A-FIG. 2E show the expression levels for ABCA1-targeting miRNAs miR-33, miR-128-1, miR-148a, miR-130b, and miR-301 b in RPE cells (either primary human RPE cells or C57BL/6J mouse RPE cells). FIG. 2A is a chart showing the longitudinal study of the expression levels of miR-33 in RPE cells from aging C57BL/6J mice. FIG. 2B is a chart showing the longitudinal study of the expression levels of miR-128-1 in RPE cells from aging C57BL/6J mice. FIG. 2C is a chart showing the longitudinal study of the expression levels of miR-148a in RPE cells from aging C57BL/6J mice. FIG. 2D is a chart showing the longitudinal study of the expression levels of miR-130b in RPE cells from aging C57BL/6J mice. FIG. 2E is a chart showing the expression levels of miR-33a, miR-33b, miR-128-1, miR-148a, miR-301b, and U6 in human primary RPE cells. FIG. 2F is an image of a western blot demonstrating ABCA1 and α-tub expression levels in primary human RPE cells post-transfection with control, anti-miR-33a, or anti-miR-33b ASO. pC: precursor scrambled control miR, aC: anti-miR control.

FIG. 3A-FIG. 3F show anti-miR-33 ASO treatment reduced cholesterol accumulation in RPE cells and attenuated retinal immune cell infiltration in mice. FIG. 3A shows serum cholesterol and triglyceride levels were measured in mice that were fed a high-fat/cholesterol diet for four weeks prior to and during subcutaneous injections of scrambled control LNA ASO or anti-miR-33 LNA ASO. FIG. 3B shows Abca1, Prkaa1, Cpt1a, and Sik1 mRNA levels were measured by quantitative RT-PCR in RPE cells isolated from mice that were fed a high-fat/cholesterol diet and then injected with scrambled control LNA ASO or anti-miR-33 LNA ASO. FIG. 3C shows immunofluorescence staining of Abca1 was performed on retinal sections of mice that were fed a high-fat/cholesterol diet and treated with scrambled control LNA ASO or anti-miR-33 LNA ASO (n=4). FIG. 3D shows retinal sections from mice that were fed a high-fat/cholesterol diet and injected with scrambled control LNA ASO or anti-miR-33 LNA ASO were stained with filipin III to investigate cholesterol accumulation (n≥8). R1-close to optic nerve head, R2-center, R3-periphery. FIG. 3E shows electron microscopy revealed RPE, Bruch's membrane (BrM), and choroid of mice that were fed a high-fat/cholesterol diet and then treated with scrambled control LNA ASO or anti-miR-33 LNA ASO (n=3). FIG. 3F shows retinal sections from high-fat/cholesterol diet fed mice that were injected with scrambled control LNA ASO or anti-miR-33 LNA ASO were immunostained against Iba1 and DAPI and the number of Iba1 positive cells infiltrating the RPE layer/retinal section were quantified. Arrows in (FIG. 3F) indicate Iba1 positive cell above the RPE cell layer. POS, photoreceptor outer segments. Scale bars: (FIG. 3C), (FIG. 3D), and (FIG. 3F) are 15 μm and (FIG. 3E) is 500 nm. All error bars represent ±S.E.M. Statistical differences between scrambled control LNA ASO and anti-miR-33 LNA ASO injected mice were calculated by unpaired t test. *P<0.05, **P<0.01, ***P<0.001

FIG. 4A-FIG. 4F show anti-miR-33 ASO treatment increased miR-33 target gene expression levels and ABCA1 protein localization in non-human primate RPE cell layer. FIG. 4A shows plasma HDL-cholesterol and total cholesterol were measured in NHPs fed a high-fat/cholesterol diet for 20 months and then switched to a regular chow diet and injected with anti-miR-33 ASO or vehicle for six weeks (n=12 per group). FIG. 4B shows expression levels of ABCA1, PRKAA1, CPT1A, CROT, SIRT6, and SIK1 were measured by quantitative RT-PCR in RPE cells isolated from NHPs injected with anti-miR-33 ASO or vehicle for six weeks (n≤5). mRNA expression levels were normalized to PPIH or HPRT1. FIG. 4D shows retinal cryosections prepared from NHPs that were treated with vehicle or anti-miR-33 ASO by subcutaneous injections were immunostained for ABCA1 and DAPI nuclear stain (n=5). FIG. 4E shows retinal sections of NHPs injected with anti-miR-33 ASO or vehicle for six weeks were stained with filipin III to label unesterified cholesterol (n=9). FIG. 4F shows retinal sections of NHPs that were injected with anti-miR-33 ASO or vehicle for six weeks were pretreated with cholesterol esterase and then stained with filipin III to label esterified cholesterol. (FIG. 4E and FIG. 4F) Four regions (R1-4) from the fovea to the periphery shown in (FIG. 4C) chosen to quantify filipin III staining in the RPE cell layer of vehicle- or anti-miR-33 ASO-treated NHP retinal sections. Arrow in (FIG. 4C) points to fovea. Scale bar in (FIG. 4D) is 10 μm and (E and F) is 50 μm. All error bars represent ±S.E.M. Statistical differences between vehicle- and anti-miR-33 ASO-injected NHPs were calculated by unpaired t test. *P<0.05, **P<0.01, ***P<0.001.

FIG. 5 shows anti-miR-33 ASO treatment reduced abnormal RPE cytoskeletal organization in the RPE cell layer of non-human primates fed a high-fat/cholesterol diet. RPE flatmounts prepared from NHPs that received subcutaneous injections of vehicle or anti-miR-33 ASO for six weeks were stained with phalloidin, examined for RPE cytoskeletal organization and then RPE cell size was quantified and segmented in the area closer to the optic nerve head (ONH), center, and the periphery. Arrows in the top panel indicates enlarged RPE cells (n≤7). Scale bars: 100 μm. All error bars represent ±S.E.M. Statistical differences between vehicle- and anti-miR-33 ASO-injected NHPs were calculated by unpaired t test. *P<0.05, **P<0.01, ***P<0.001.

FIG. 6A-FIG. 6B show anti-miR-33 ASO treatment reduced immune cell infiltration in RPE-photoreceptor and RPE layers. FIG. 6A shows IBA1 (magenta) and superimposed DAPI (blue) staining revealing IBA1 positive cells in the RPE-photoreceptor and sub-RPE layers in vehicle-treated NHP retinal sections, while low IBA1 positive staining is seen in the sub-RPE-choroid layer of anti-miR-33 ASO-injected NHP retinal sections. FIG. 6B is an ImageJ 3D reconstruction revealing IBA1 (magenta) and DAPI (blue) stained retinal sections from vehicle- and anti-miR-33 ASO-treated NHPs. Scale bar in (FIG. 6A) is 10 μm. (OS) refers to outer segment and (IS) to inner segments of photoreceptor cells.

FIG. 7 is series of charts showing circulating alanine aminotransferase (ALT), aspartate aminotransferase (AST), bilirubin, and uric acid in mice following the anti-miR-33 LNA ASO treatment.

FIG. 8A and FIG. 8B are series of images showing infiltration of Iba1 positive microglial cells into the photoreceptor nuclear layer in the control LNA ASO-treated mice but not in miR-33 LNA ASO-treated mice.

FIG. 9A is a series of charts showing LDL-C levels, VLDL-C levels, and triglyceride levels in the treatment groups relative to baseline.

FIG. 9B is a series of charts showing ALT levels, AST levels, creatinine levels, and blood urea nitrogen levels in the treatment groups relative to baseline.

FIG. 10A-FIG. 10B are a series of charts showing SREBF2, SREBF1, miR-33a, and miR-33b expression levels NHP RPE cells from animals receiving anti-miR-33 ASO or a vehicle.

FIG. 11A is a series of images showing ABCA1 protein levels in the RPE cell layer of anti-miR-33 ASO-treated NHPs as compared to the vehicle-treated NHP from fovea to periphery.

FIG. 11B is a series of images showing expression pattern of ABCA1 protein in the neural retina of vehicle- or anti-miR-33-treated NHP retinal sections.

FIG. 12A is a series of images showing the expression of APOE in the RPE of anti-miR-33 ASO-treated group in comparison to vehicle-treated group

FIG. 12B is a series of images showing the APOE staining in the neural retina of anti-miR-33 ASO-treated or vehicle-treated groups.

FIG. 13A-FIG. 13B are a series of images showing filipin III stained NHP retinal sections of vehicle or anti-miR-33 ASO-treated groups.

### DETAILED DESCRIPTION

In general, the invention relates to oligonucleotides, rAAV particles, pharmaceutical compositions, and methods that may be useful in the treatment of age-related macular degeneration (e.g., dry age-related macular degeneration). The treatment of age-related macular degeneration may involve inhibition of an miR-33 target nucleic acid.

The invention is based, in part, on the invention of miR-33 inhibitors (e.g., oligonucleotides targeting the an miR-33 target nucleic acid) for use in the treatment of age-related macular degeneration (AMD). In particular, the miR-33 family of microRNAs was found to be responsible for pathological cholesterol accumulation and inflammation in the retina, hallmarks of AMD, the leading cause of blindness in the elderly. There are two forms of AMD, wet and dry. The wet form is characterized by abnormal angiogenesis in the retina, termed choroidal neovascularization (CNV). Direct ocular injection with antibodies targeting VEGF exhibit some efficacy in slowing the wet form of AMD, however, importantly, there is no approved treatment for dry AMD, which is characterized by cholesterol accumulation in the retina in so called “drusen” deposits, as well as inflammation that causes death of retinal pigment epithelial (RPE) cells, leading to what is termed geography atrophy and blindness. The studies presented herein demonstrate that inhibition of miR-33 in mouse and non-human primate models of dry AMD results in amelioration of pathologies associated with this prevalent disease.

Antisense Oligonucleotide

In one approach, the invention provides a single-stranded oligonucleotide having a nucleobase sequence with at least 7 contiguous nucleobases complementary to an equal-length portion within an miR-33 target nucleic acid. This approach is typically referred to as an antisense approach, and the corresponding oligonucleotides may be referred to as antisense oligonucleotides (ASO). Without wishing to be bound by theory, this approach involves hybridization of an oligonucleotide of the invention to an miR-33 target nucleic acid (e.g., pri-miR-33a, pre-miR-33a, miR-33a, pre-miR-33b, pri-miR-33b, and miR-33b), followed by ribonuclease H (RNase H) mediated cleavage of the miR-33 target nucleic acid. Alternatively and without wishing to be bound by theory, this approach involves hybridization of an oligonucleotide of the invention to an miR-33 target nucleic acid (e.g., pri-miR-33a, pre-miR-33a, miR-33a, pre-miR-33b, pri-miR-33b, and miR-33b), thereby sterically blocking the miR-33 target nucleic acid from binding to the targets of miR-33. In some embodiments, the single-stranded oligonucleotide may be delivered to a subject as a double stranded oligonucleotide, where the oligonucleotide of the invention is hybridized to another oligonucleotide (e.g., an oligonucleotide having a total of 12 to 30 nucleotides).

An antisense oligonucleotide may be, e.g., a unimer or a gapmer. Gapmers are oligonucleotides having an RNase H recruiting region (gap) flanked by a 5′ wing and 3′ wing, each of the wings including at least one affinity enhancing nucleoside (e.g., 1, 2, 3, or 4 affinity enhancing nucleosides). In certain embodiments, each wing includes 1-5 nucleosides. In some embodiments, each nucleoside of each wing is a modified nucleoside. In particular embodiments, the gap includes 7-15 nucleosides. Typically, the gap region includes a plurality of contiguous, unmodified deoxyribonucleotides. For example, all nucleotides in the gap region are unmodified deoxyribonucleotides (2′-deoxyribofuranose-based nucleotides). In some embodiments, an antisense oligonucleotide of the invention (e.g., a single-stranded oligonucleotide of the invention) is a gapmer. Unimers are oligonucleotides having all nucleotides with a common modification. For example, all nucleotides in a unimer may be independently, e.g., bridged nucleic acids, e.g., locked nucleic acids or ethylene bridged nucleic acids. Preferably, all nucleotides in a unimer are independently locked nucleic acids. Without wishing to be bound by theory, it is believed that unimers (e.g., those including bridged nucleic acids) may inhibit an miR-33 target nucleic acid sterically, e.g., without recruiting RNase H.

An antisense oligonucleotide may include a total of at least 7 contiguous nucleotides (e.g., 7, 8, 9, 10, 11, 12, 13, 14, 15, 16, 17, 18, 19, 20, 21, 22, 23, or 24 contiguous nucleotides). Preferably, the antisense oligonucleotide includes a total of fewer than 30 contiguous nucleotides (e.g., fewer than 25, 26, 27, 28, 29, 30, 31, 32, 33, 34, 35, 36, 37, 38, 39, or 40 contiguous nucleotides). An antisense oligonucleotide described herein may include 6, 7, 8, 9, 10, 11, 12, 13, 14, 15, 16, 17, 18, 19, 20, or more contiguous nucleotides complementary to an miR-33 target nucleic acid. In some embodiments, the antisense oligonucleotide has a nucleotide sequence that is complementary to an equal length portion of an miR-33 target nucleic acid. Thus, an antisense oligonucleotide may include a total of 6, 7, 8, 9, 10, 11, 12, 13, 14, 15, 16, 17, 18, 19, 20 or more contiguous nucleotides that are complementary to an miR-33 target nucleic acid.

Interfering RNA (RNAi)

An interfering RNA includes an antisense sequence that is complementary to a target RNA and a sense sequence that is the reverse complement of the antisense sequence. Typically, the antisense sequence and the sense sequence are at least partially hybridized to each (the extent of hybridization may depend on, for example, the presence of overhangs). As is described herein, the target RNA is an miR-33 target nucleic acid.

The RNAi approach typically utilizes siRNA or shRNA. Without wishing to be bound by theory, this approach involves incorporation of the sense sequence into an RNA-induced silencing complex (RISC), which can identify and hybridize to an miR-33 target nucleic acid in a cell through complementarity of a portion of the sense sequence and the miR-33 target nucleic acid. Upon identification (and hybridization), RISC may either remain on the target nucleic acid thereby sterically blocking translation or cleave the target nucleic acid.

In siRNA, an antisense sequence is typically referred to as a guide strand, and a sense sequence is typically referred to as a passenger strand. Thus, an siRNA is typically a double-stranded oligonucleotide including a passenger strand hybridized to a guide strand having a nucleobase sequence with at least 8 contiguous nucleobases complementary to an equal-length portion within an miR-33 target nucleic acid. An siRNA guide strand may include a total of at least 8 contiguous nucleotides (e.g., 8, 9, 10, 11, 12, 13, 14, 15, 16, 17, 18, 19, 20, 21, 22, 23, or 24 contiguous nucleotides). Preferably, an siRNA guide strand includes a total of fewer than 30 contiguous nucleotides (e.g., fewer than 25, 26, 27, 28, 29, 30, 31, 32, 33, 34, 35, 36, 37, 38, 39, or 40 contiguous nucleotides). An siRNA passenger strand may include a total of at least 8 contiguous nucleotides (e.g., 8, 9, 10, 11, 12, 13, 14, 15, 16, 17, 18, 19, 20, 21, 22, 23, or 24 contiguous nucleotides). Preferably, an siRNA passenger strand includes a total of fewer than 30 contiguous nucleotides (e.g., fewer than 25, 26, 27, 28, 29, 30, 31, 32, 33, 34, 35, 36, 37, 38, 39, or 40 contiguous nucleotides). The siRNA may include at least one 3′-overhang (e.g., 1, 2, 3, or 4 nucleotide-long overhang; e.g., UU overhang). In particular embodiments, the siRNA is a blunt. In some embodiments, the siRNA includes two 3′-overhangs (e.g., 1, 2, 3, or 4 nucleotide-long overhang; e.g., UU overhang). In some embodiments, the guide strand of the siRNA includes a region of complementarity with a region of at least 8 (e.g., at least 8, at least 9, at least 10, at least 11, at least 12, at least 13, at least 14, at least 15, at least 16, at least 17, at least 18, at least 19, at least 20, or at least 21, e.g., 8, 9, 10, 11, 12, 13, 14, 15, 16, 17, 18, 19, 20, or 21) contiguous nucleotides of an miR-33 target nucleic acid. An siRNA guide strand described herein may include 6, 7, 8, 9, 10, 11, 12, 13, 14, 15, 16, 17, 18, 19, 20, or more contiguous nucleotides complementary to an miR-33 target nucleic acid. In some embodiments, an siRNA guide strand has a nucleotide sequence that is complementary to an equal length portion of an miR-33 target nucleic acid. Thus, an siRNA guide strand may include a total of 6, 7, 8, 9, 10, 11, 12, 13, 14, 15, 16, 17, 18, 19, 20 or more contiguous nucleotides that are complementary to an miR-33 target nucleic acid.

In shRNA, the antisense sequence and the sense sequence are typically separated by a spacer or loop sequence. A spacer or loop can be of a sufficient length to permit the antisense and sense sequences to anneal and form a double-stranded structure (or stem). The spacer can then be cleaved away to form a double-stranded RNA (and, optionally, subsequent processing steps that may result in addition or removal of one, two, three, four, or more nucleotides from the 3′ end and/or the 5′ end of either or both strands). In some embodiments, the stem of the shRNA includes 19-29 basepairs, and the loop includes 4-8 nucleotides, optionally with a dinucleotide overhang at the 3′ end of the shRNA. In some embodiments, the stem of the shRNA includes a region of complementarity with a region of at least 8 (e.g., at least 8, at least 9, at least 10, at least 11, at least 12, at least 13, at least 14, at least 15, at least 16, at least 17, at least 18, at least 19, at least 20, or at least 21, e.g., 8, 9, 10, 11, 12, 13, 14, 15, 16, 17, 18, 19, 20, or 21) contiguous nucleotides of an miR-33 target nucleic acid.

Adeno-Associated Viruses

Short hairpin RNA (shRNA) may be delivered to a subject in need thereof using a recombinant adeno-associated viral (AAV). Any AAV-mediated delivery approach suitable for targeting the eye may be used. The AAV particle described herein may include a nucleic acid vector that includes a heterologous nucleic acid region including a sequence that encodes an interfering RNA including a region (e.g., at least 8, at least 9, at least 10, at least 11, at least 12, at least 13, at least 14, at least 15, at least 16, at least 17, at least 18, at least 19, at least 20, or at least 21, e.g., 8, 9, 10, 11, 12, 13, 14, 15, 16, 17, 18, 19, 20, or 21 contiguous nucleotides) complementary to an miR-33 target nucleic acid.

In some embodiments of the aspects and embodiments described above, the AAV viral particle comprises an AAV1, AAV2, AAV3, AAV4, AAV5, AAV6 (e.g., a wild-type AAV6 capsid, or a variant AAV6 capsid such as ShH10, as described in US 20120164106), AAV7, AAV8, AAVrh8, AAVrh8R, AAV9 (e.g., a wild-type AAV9 capsid, or a modified AAV9 capsid as described in US 20130323226), AAV10, AAVrh10, AAV11, AAV12, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in U.S. PG Pub. 2012/0066783), AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, rAAV2/HBoV1 capsid, or an AAV capsid described in U.S. Pat. No. 8,283,151 or International Publication No. WO 2003042397. In some embodiments, the AAV viral particle comprises an AAV capsid comprising an amino acid substitution at one or more of positions R484, R487, K527, K532, R585 or R588, numbering based on VP1 of AAV2. In further embodiments, an AAV particle comprises capsid proteins of an AAV serotype from Clades A-F. In some embodiments, the rAAV viral particle comprises an AAV serotype 2 capsid. In further embodiments, the AAV serotype 2 capsid comprises AAV2 capsid protein comprising a R471A amino acid substitution, numbering relative to AAV2 VP1. In some embodiments, the vector comprises AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats (ITRs). In some embodiments, the vector comprises AAV serotype 2 ITRs. In some embodiments, the AAV viral particle comprises one or more ITRs and capsid derived from the same AAV serotype. In other embodiments, the AAV viral particle comprises one or more ITRs derived from a different AAV serotype than capsid of the rAAV viral particles. In some embodiments, the rAAV viral particle comprises an AAV2 capsid, and wherein the vector comprises AAV2 ITRs. In further embodiments, the AAV2 capsid comprises AAV2 capsid protein comprising a R471A amino acid substitution, numbering relative to AAV2 VP1.

The interfering RNA (e.g., shRNA) may be operably linked to and under expression control of a promoter sequence as described herein. The promoter may be capable of expressing the interfering RNA (e.g., shRNA), e.g., in the eye of the subject. Non-limiting examples of promoters include a hybrid chicken β-actin (CBA) promoter and an RNA polymerase III promoter.

The vector may include, e.g., AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats (ITRs). In some embodiments, the vector includes AAV serotype 2 ITRs. In some embodiments, the rAAV particle includes one or more ITRs and capsid derived from the same AAV serotype. In other embodiments, the rAAV particle includes one or more ITRs derived from a different AAV serotype than capsid of the rAAV particles.

The rAAV particle may include, e.g., an AAV1, AAV2, AAV3, AAV4, AAV5, AAV6 (e.g., a wild-type AAV6 capsid, or a variant AAV6 capsid such as ShH10, as described in US 20120164106), AAV7, AAV8, AAVrh8, AAVrh8R, AAV9 (e.g., a wild-type AAV9 capsid, or a modified AAV9 capsid as described in US 20130323226), AAV10, AAVrh10, AAV11, AAV12, a tyrosine capsid mutant, a heparin binding capsid mutant, an AAV2R471A capsid, an AAVAAV2/2-7m8 capsid, an AAV DJ capsid (e.g., an AAV-DJ/8 capsid, an AAV-DJ/9 capsid, or any other of the capsids described in US 20120066783), AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, rAAV2/HBoV1 capsid, or an AAV capsid described in U.S. Pat. No. 8,283,151 or International Publication No. WO 2003042397. In some embodiments, the AAV viral particle includes an AAV capsid including an amino acid substitution at one or more of positions R484, R487, K527, K532, R585 or R588, numbering based on VP1 of AAV2. In further embodiments, a rAAV particle includes capsid proteins of an AAV serotype from Clades A-F. In some embodiments, the rAAV viral particle includes an AAV serotype 2 capsid. In further embodiments, the AAV serotype 2 capsid includes AAV2 capsid protein including a R471A amino acid substitution, numbering relative to AAV2 VP1. In some embodiments, the vector includes AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats (ITRs). In some embodiments, the vector includes AAV serotype 2 ITRs. In some embodiments, the AAV viral particle includes one or more ITRs and capsid derived from the same AAV serotype. In other embodiments, the AAV viral particle includes one or more ITRs derived from a different AAV serotype than capsid of the rAAV viral particles.

An AAV vector which encodes an interfering RNA can be generated using methods known in the art, using standard synthesis and recombinant methods.

Dosing and Administration

In some embodiments, an oligonucleotide, composition including an oligonucleotide, or rAAV particle described herein is administered by intraocular injection, intravitreal injection, subretinal injection, topical application, implantation, intraperitoneal injection, intramuscular injection, subcutaneous injection, or intravenous injection of the subject. Methods of pharmaceutical composition delivery to the eye are known in the art, e.g., are described in US 20170304465, the disclosure of which is incorporated herein by reference.

In some embodiments, an oligonucleotide or a rAAV particle described herein may be administered once daily, every other day, once weekly, twice weekly, three times weekly, four times weekly, biweekly, monthly, bimonthly, quarterly, every 6 months, or annually. Preferably, an oligonucleotide or a rAAV particle described herein is administered once weekly to once monthly.

Methods of subretinal delivery are known in the art. For example, see WO 2009/105690, incorporated herein by reference. Briefly, the general method for delivering an oligonucleotide, composition including it, or rAAV particle described herein to the subretina of the macula and fovea may be illustrated by the following brief outline. This example is merely meant to illustrate certain features of the method, and is in no way meant to be limiting.

Generally, an oligonucleotide or rAAV particle described herein can be delivered in the form of a composition injected intraocularly (e.g., subretinally) under direct observation using an operating microscope. This procedure may involve vitrectomy followed by injection of an oligonucleotide solution of an rAAV particle suspension using a fine cannula through one or more small retinotomies into the subretinal space.

Briefly, an infusion cannula can be sutured in place to maintain a normal globe volume by infusion (of e.g., saline) throughout the operation. A vitrectomy is performed using a cannula of appropriate bore size (for example 20 to 27 gauge), wherein the volume of vitreous gel that is removed is replaced by infusion of saline or other isotonic solution from the infusion cannula. The vitrectomy is advantageously performed because (1) the removal of its cortex (the posterior hyaloid membrane) facilitates penetration of the retina by the cannula; (2) its removal and replacement with fluid (e.g., saline) creates space to accommodate the intraocular injection of vector, and (3) its controlled removal reduces the possibility of retinal tears and unplanned retinal detachment.

In some embodiments, an oligonucleotide or rAAV particle described herein is directly injected into the subretinal space outside the central retina, by utilizing a cannula of the appropriate bore size (e.g., 27-45 gauge), thus creating a bleb in the subretinal space. In other embodiments, the subretinal injection of an oligonucleotide or rAAV particle described herein is preceded by subretinal injection of a small volume (e.g., about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of an oligonucleotide or rAAV particle described herein to the subretinal space (by defining the plane of injection prior to the delivery of an oligonucleotide or rAAV particle described herein), and minimize possible administration into the choroid and the possibility of injection or reflux into the vitreous cavity.

Intraocular administration of an oligonucleotide or rAAV particle described herein and/or the initial small volume of fluid can be performed using a fine bore cannula (e.g., 27-45 gauge) attached to a syringe. In some embodiments, the plunger of this syringe may be driven by a mechanized device, such as by depression of a foot pedal. The fine bore cannula is advanced through the sclerotomy, across the vitreous cavity and into the retina at a site pre-determined in each subject according to the area of retina to be targeted (but outside the central retina). Under direct visualization the vector suspension is injected mechanically under the neurosensory retina causing a localized retinal detachment with a self-sealing non-expanding retinotomy. As noted above, an oligonucleotide or rAAV particle described herein can be either directly injected into the subretinal space creating a bleb outside the central retina or the vector can be injected into an initial bleb outside the central retina, causing it to expand (and expanding the area of retinal detachment). In some embodiments, the injection of an oligonucleotide or rAAV particle described herein is followed by injection of another fluid into the bleb.

Without wishing to be bound by theory, the rate and location of the subretinal injection(s) can result in localized shear forces that can damage the macula, fovea and/or underlying RPE cells. The subretinal injections may be performed at a rate that minimizes or avoids shear forces. In some embodiments, an oligonucleotide or rAAV particle described herein is injected over about 15-17 minutes. In some embodiments, an oligonucleotide or rAAV particle described herein is injected over about 17-20 minutes. In some embodiments, an oligonucleotide or rAAV particle described herein is injected over about 20-22 minutes. In some embodiments, an oligonucleotide or rAAV particle described herein is injected at a rate of about 35 to about 65 μl/min. In some embodiments, an oligonucleotide or rAAV particle described herein is injected at a rate of about 35 μl/min. In some embodiments, an oligonucleotide or rAAV particle described herein is injected at a rate of about 40 μl/min. In some embodiments, an oligonucleotide or rAAV particle described herein is injected at a rate of about 45 μl/min. In some embodiments, an oligonucleotide or rAAV particle described herein is injected at a rate of about 50 μl/min. In some embodiments, an oligonucleotide or rAAV particle described herein is injected at a rate of about 55 μl/min. In some embodiments, an oligonucleotide or rAAV particle described herein is injected at a rate of about 60 μl/min. In some embodiments, an oligonucleotide or rAAV particle described herein is injected at a rate of about 65 μl/min. One of ordinary skill in the art would recognize that the rate and time of injection of the bleb may be directed by, for example, the volume of the pharmaceutical composition or size of the bleb necessary to create sufficient retinal detachment to access the cells of central retina, the size of the cannula used to deliver the pharmaceutical composition, and the ability to safely maintain the position of the cannula of the invention.

In some embodiments of the invention, the volume of the composition injected to the subretinal space of the retina is more than about any one of 1 μl, 2 μl, 3 μl, 4 μl, 5 μl, 6 μl, 7 μl, 8 μl, 9 μl, 10 μl, 15 μl, 20 μl, 25 μl, 50 μl, 75 μl, 100 μl, 200 μl, 300 μl, 400 μl, 500 μl, 600 μl, 700 μl, 800 μl, 900 μl, or 1 mL, or any amount therebetween.

An effective concentration of a recombinant adeno-associated virus carrying a vector as described herein ranges between about 10⁸ and 10¹³ vector genomes per milliliter (vg/mL). The rAAV infectious units are measured as described in McLaughlin et al., J. Virol. 1988, 62: 1963. In one embodiment, the concentration ranges between 10⁹ and 10¹³ vg/mL. In another embodiment, the effective concentration is about 1.5×10¹¹ vg/mL. In one embodiment, the effective concentration is about 1.5×10¹⁰ vg/mL. In another embodiment, the effective concentration is about 2.8×10¹¹ vg/mL. In another embodiment, the effective concentration is about 5×10¹¹ vg/mL. In yet another embodiment, the effective concentration is about 1.5×10¹² vg/mL. In another embodiment, the effective concentration is about 1.5×10¹³ vg/mL.

It is desirable that the lowest effective dosage (total genome copies delivered) of virus be utilized in order to reduce the risk of undesirable effects, such as toxicity, and other issues related to administration to the eye. An effective dosage of a recombinant adeno-associated virus carrying a trans-splicing molecule as described herein ranges between about 10⁸ and 10¹³ vector genomes (vg) per dose (i.e, per injection). In one embodiment, the dosage ranges between 10⁹ and 10¹³ vg. In another embodiment, the effective dosage is about 1.5×10¹¹ vg. In another embodiment, the effective dosage is about 5×10¹¹ vg. In one embodiment, the effective dosage is about 1.5×10¹⁰ vg. In another embodiment, the effective dosage is about 2.8×10¹¹ vg. In yet another embodiment, the effective dosage is about 1.5×10¹² vg. In another embodiment, the effective concentration is about 1.5×10¹³ vg. Still other dosages in these ranges or in other units may be selected by the attending physician, taking into account the physical state of the subject being treated, including the age of the subject; the composition being administered, and the particular disorder.

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 350 μL. In another embodiment, the volume is about 400 μL In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1,000 μL.

In one embodiment, the volume and concentration of the rAAV composition is selected so that only certain anatomical regions having target cells are impacted. In another embodiment, the volume and/or concentration of the rAAV composition is a greater amount, in order reach larger portions of the eye. Similarly dosages are adjusted for administration to other organs.

The following examples are meant to illustrate the invention. They are not meant to limit the invention in any way.

### EXAMPLES

Defective cholesterol/lipid homeostasis is linked to neurodegenerative conditions including AMD (1). In particular, age-related deposition of cholesterol and cholesterol-containing “drusen” in the RPE and sub-RPE layers is strongly associated with the development of AMD (2, 3). Moreover, genome-wide association studies (GWAS) of genetic risk factors linked to AMD have identified single nucleotide polymorphisms near genes involved in cholesterol and lipid regulation such as ABCA1, APOE, CETP, and LIPC (4-7). Studies with ApoE4 knock-in mice and ApoB100/Lldr^{-/-} mice also demonstrate that cholesterol deposition in the RPE layer induces AMD-like pathology (8, 9), providing further support for the hypothesis that abnormal cholesterol accumulation in the retina represents a prominent pathological feature. There is currently no treatment for dry AMD, particularly late stage geographic atrophy, and targeting mechanisms linked to cholesterol accumulation may be a viable therapeutic strategy in dry AMD (10). In support of this concept, a recent pilot study of 23 patients has shown that high doses of atorvastatin (80 mg) led to the disappearance of drusen and improved visual acuity in at least 10 patients (10).

RPE cells are key regulators of cholesterol homeostasis in the retina (11). Mice that lack ABCA1 along with ABCG1 in the RPE layer develop AMD-like pathology that includes cholesterol accumulation, RPE and photoreceptor degeneration, and inflammation (11). Moreover, a previous study revealed that expression of ABCA1 is downregulated during aging in macrophages, and linked the corresponding decreased cholesterol efflux capacity in aging macrophages to elevated retinal inflammation and choroidal neovascularization (CNV) (12), a pathological hallmark of the “wet” form of AMD that is characterized by new blood vessel growth.

MicroRNAs (miRNAs) are short (˜22 nucleotides) non-coding RNAs with diverse functions in development, metabolism and disease (13). Aberrant expression or function of miRNAs has been linked to a number of diseases, and inhibition of several disease-associated miRNAs with anti-miR antisense oligonucleotides (ASOs) has recently been explored as a therapeutic intervention (14,15).

We have for a number of years investigated conserved mechanisms by which the sterol regulatory element-binding protein (SREBP) family of transcription factors governs cholesterol/lipid and metabolic homeostasis (16-20). We and others recently discovered that the human SREBP genes surprisingly harbor intronic miRNAs, miR-33a/b (21, 22). Intriguingly, our studies and those of others have revealed that miR-33a/b act to modulate several interconnected metabolic circuits, while also cooperating with the SREBP transcription factors to promote elevated intracellular cholesterol/fatty acids, and other lipids. Importantly, injection of ASOs directed against miR-33 results in significantly increased hepatic and macrophage ABCA1 expression and elevated circulating HDL-C in mice and non-human primates and decrease atherosclerosis in Ldlr^{-/-} and Apoe^{-/-} mice (21-25).

We have also carried out a systematic analysis of GWAS involving >188,000 individuals, associating common SNPs with abnormal plasma lipids, resulting in the identification of 69 miRNAs. Two miRNAs, miR-128-1 and miR148a, emerged from these analyses as the top microRNAs with predicted targets involved in cholesterol/lipid and metabolic homeostasis, including ABCA1 (26). As with miR-33, we found that these miRNAs are also critical regulators of ABCA1-dependent cholesterol efflux from macrophages. These studies together provide evidence that miRNAs may serve as key regulators of cholesterol/lipid homeostasis, with important implications for cholesterol/lipid disorders.

Importantly, a recent study found that miR-33 expression is elevated in aging mouse macrophages, and linked this to increased inflammation and CNV in the mouse eye (12). However, the potential roles of cholesterol/lipid-regulating miRNAs in animal models recapitulating aspects of dry AMD have not been explored. Taken together with the apparent cholesterol accumulation in AMD and strong genetic connections with cholesterol regulators, this then provides the impetus to explore more deeply the potential broader functional association of miRNAs with dry AMD. Here, we describe our evaluation of miRNA regulation of ABCA1 and high-fat/cholesterol diet-induced cholesterol accumulation and AMDlike pathologies in the eye of mice and non-human primates.

Results

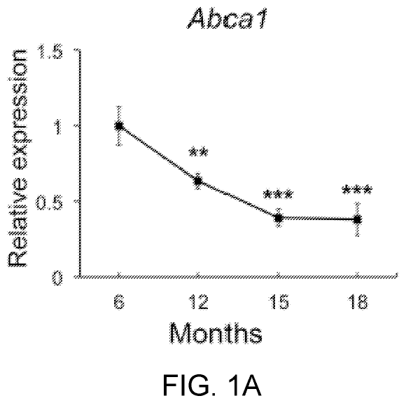

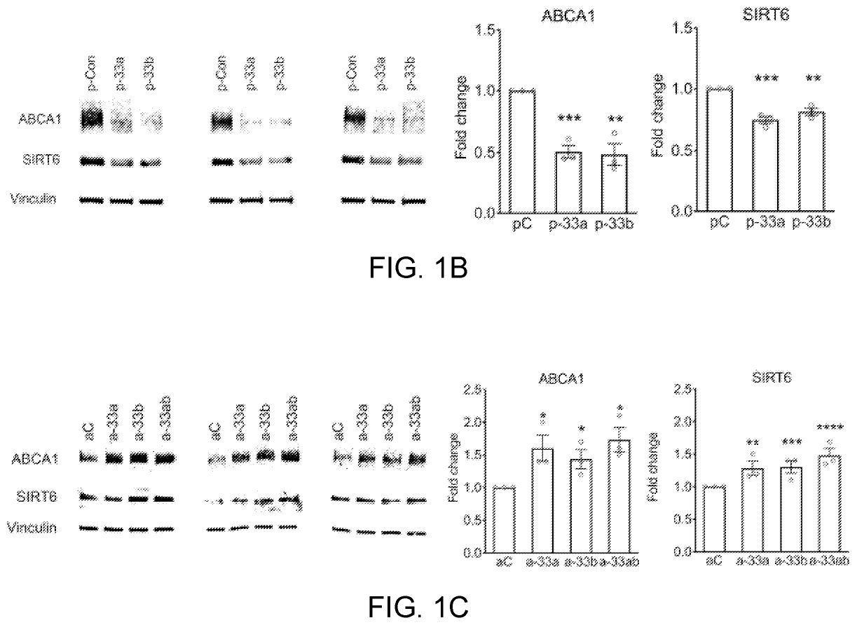

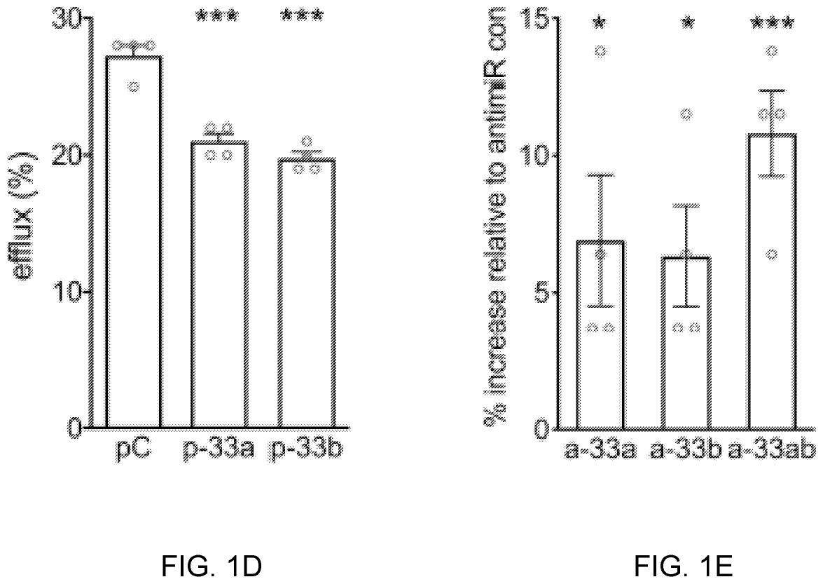

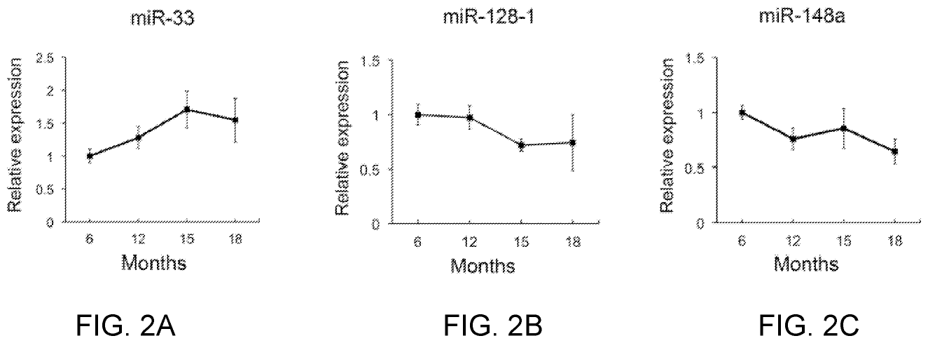

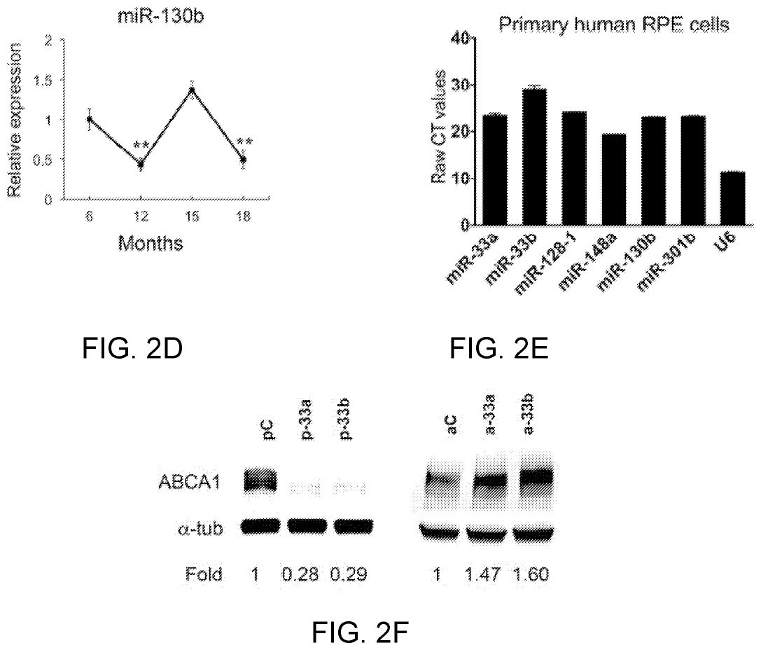

We first analyzed by real-time quantitative PCR whether the expression of Abca1 in RPE cells freshly isolated from C57BL/6J mice is altered with aging. The results revealed that the level of Abca1 mRNA was markedly decreased in RPE cells as mice age (FIG. 1A). Next, we showed that the ABCA1-targeting miRNAs miR-33, miR-128-1, miR-148a, miR-130b, and miR-301b are expressed in primary human RPE cells, but that only miR-33 expression was increased in RPE cells of aging mice (FIG. 2A-E). We therefore focused follow up studies on miR-33.

We first investigated whether miR-33 affects ABCA1 expression in primary human RPE cells, and then further characterized the role of miR-33 in RPE cholesterol efflux using the human RPE-derived cell line ARPE-19. We found that introduction of excess miR-33a and/or miR-33b isoforms resulted in decreased ABCA1 levels in primary human RPE and ARPE-19 cells (FIG. 2F and FIG. 1B), whereas inhibition of endogenous miR-33a or miR-33b using anti-miR ASOs in primary human RPE and ARPE-19 cells increased ABCA1 levels (FIG. 2F and FIG. 1C). We then determined if the elevated expression of miR-33a and miR-33b would have a functional effect on cholesterol handling by the RPE. Transfection of precursor miR-33a or miR-33b significantly reduced cholesterol efflux to carrier ApoA1 lipoprotein as compared with scrambled precursor miR control (FIG. 1D). The inhibition of endogenous miR-33a and miR-33b with anti-miR-33a/b ASOs produced an additive positive effect on ABCA1 expression (FIG. 1C) and significantly improved cholesterol efflux (P=0.001) in ARPE-19 cells as compared to anti-miR-33a (P=0.04), antimiR-33b (P=0.02), or control ASO (FIG. 1E). In addition to ABCA1, miR-33 has been shown to target the lipid regulator SIRT6 in human cells (22, 27). Transfection of ARPE-19 cells with miR-33a or miR-33b precursors significantly reduced SIRT6 levels compared to scrambled miR transfected cells (FIG. 1B). Conversely, transfection of anti-miR-33a, anti-miR-33b or anti-miR-33a/b ASOs significantly increased SIRT6 levels, compared to control ASO transfected ARPE-19 cells (FIG. 1C).

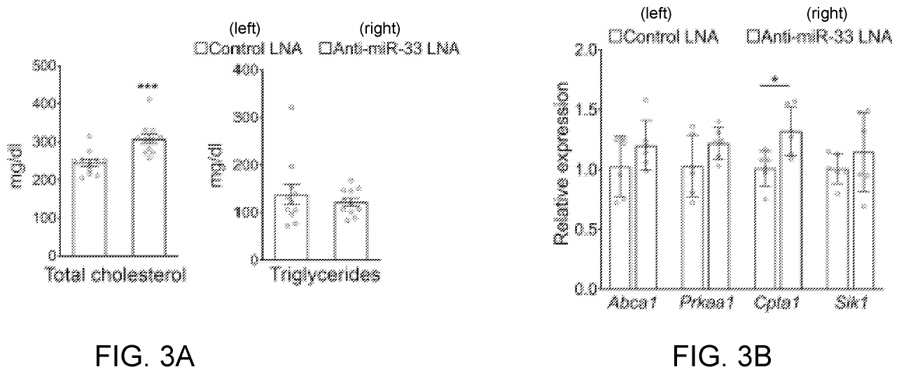

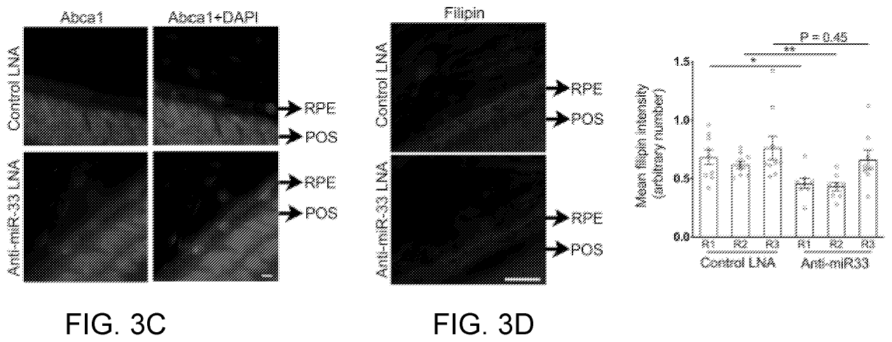

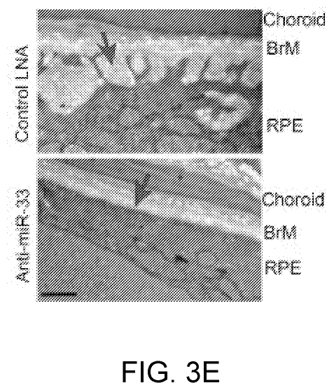

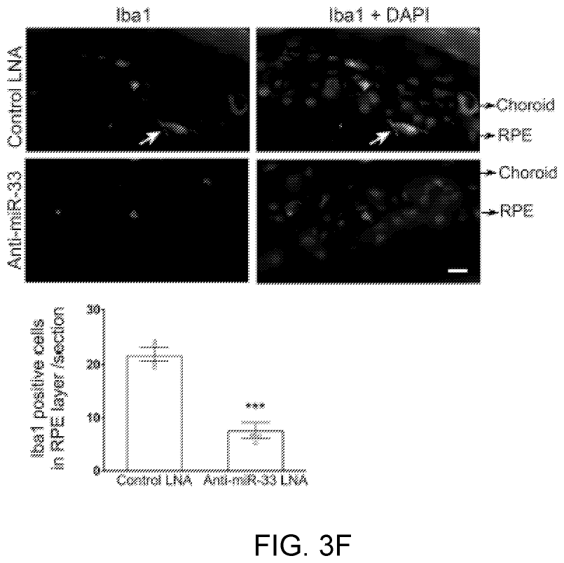

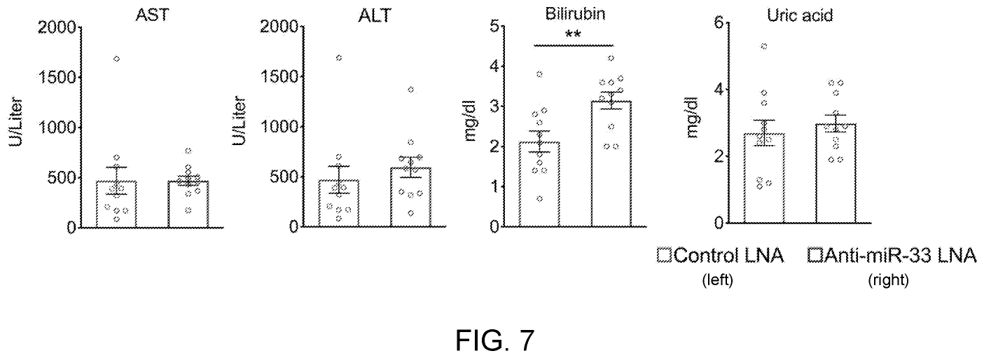

Since we observed that Abca1 gene expression was significantly decreased in the RPE cells of aging mice (FIG. 1A), we tested whether feeding 12-month old C57BL/6J male mice a high-fat/cholesterol Western-type diet (WTD) for eight weeks would result in cholesterol accumulation in the RPE layer, and whether inhibition of miR-33 by subcutaneous delivery of anti-miR-33 locked nucleic acid (LNA) ASO (22, 23) for four weeks (while on the WTD) would reduce cholesterol deposition. Consistent with previous reports (21-23), anti-miR-33 LNA ASO treatment significantly increased total serum cholesterol (predominantly HDL-C) levels as compared to mice that received scrambled control LNA ASO (FIG. 3A), without significantly affecting circulating liver enzymes alanine aminotransferase (ALT) and aspartate aminotransferase (AST), and with very moderate effects on bilirubin and creatinine (FIG. 7), suggesting the treatment was well tolerated as previously observed (21, 22). We collected RNA from RPE cells from the dissected retinas of animals that were treated with scrambled control or anti-miR-33 LNA ASO to examine the effect on miR-33 target gene expression levels, Abca1 protein localization, and cholesterol deposition in the RPE cell layer. In agreement with on-target effects of anti-miR33, expression of several miR-33 target genes (e.g., Cpt1a, Abca1, Prkaa1, and Sik1 (22, 27)) were modestly increased in the RPE of anti-miR-33 LNA ASO-treated mice compared to scrambled control LNA ASO treatment (FIG. 3B).

We next analyzed Abca1 expression in cryosections of eyecups by immunofluorescence and, consistent with the effects on Abca1 mRNA levels, we found that there was stronger Abca1 staining in the RPE cell layer, as well as in choroid blood vessels, of eyecups from animals treated with anti-miR-33 LNA ASO as compared to control LNA ASO treatment (FIG. 3C).

To study the effect of LNA ASO treatment on cholesterol deposition in the RPE layer, retinal sections were stained with the cholesterol-trophic dye filipin III. Analysis of the eyecups from animals treated with scrambled control LNA ASO showed strong filipin III staining in the RPE layer closer to the optic nerve head, center and the periphery (FIG. 3D), whereas eyecups from animals treated with anti-miR-33 LNA ASO exhibited significant reduction of filipin III staining in the RPE layer closer to the optic nerve head region and the central region, but not in the periphery (FIG. 3D). We speculate that the lesser effect of the LNA ASO treatment on cholesterol accumulation in the retina periphery might be due to the LNA ASO not effectively reaching the peripheral RPE layer as compared to the RPE cell layer closer to the optic nerve head region and the central region.

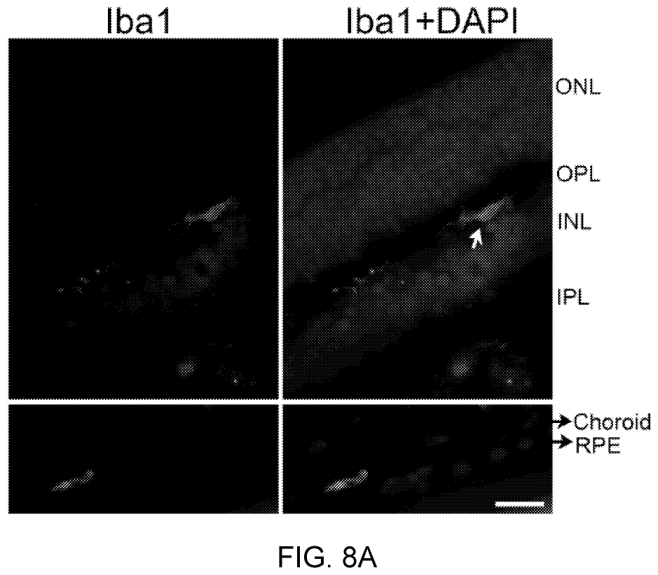

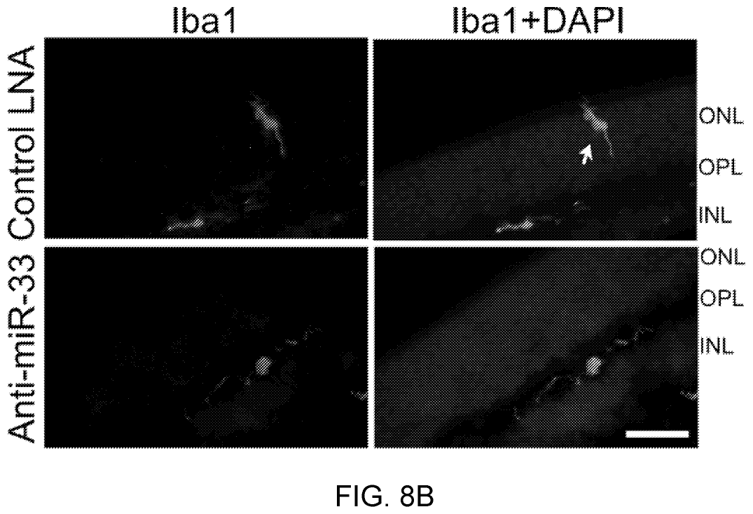

Cholesterol accumulation interferes with RPE basal infoldings and causes sub-RPE deposit formation (8). Electron microscopic imaging showed that RPE basal structures were disrupted in the retina of control LNA ASO-treated mice (FIG. 3E); in contrast, RPE basal infoldings were largely preserved in anti-miR33 LNA ASO-treated mice (FIG. 3E). Moreover, it appeared that lipid-dense deposits in the Bruch's membrane (BrM) were elevated in the retina of scrambled control LNA ASO-treated mice as compared to retinas from animal treated with anti-miR-33 LNA ASO (FIG. 3E). As AMD is associated with immune cell infiltration in the retina (28, 29), we then examined whether cholesterol accumulation in the RPE layer is linked to inflammatory cell recruitment, as judged by staining with an antibody directed against Iba1 (macrophage/microglia marker). The average number of Iba1 positive cells in the RPE cell layer of antimiR-33 LNA ASO-treated mice was markedly and significantly lower (8±3 cells/retinal section) as compared with scrambled control LNA ASO-treated mice (22±2 cells/retinal section) (FIG. 3F). In addition, infiltration of Iba1 positive microglial cells into the photoreceptor nuclear layer was observed in the control LNA ASO-treated mice but not in miR-33 LNA ASO-treated mice (FIGS. 8A and 8B), consistent with a potent anti-inflammatory effect of anti-miR-33 treatment in the eye of aging WTD-fed mice. These results led us to further investigate the therapeutic value of miR-33 inhibition in reducing cholesterol accumulation and inflammation in WTD-fed male non-human primates (NHPs, Cynomolgus monkeys).

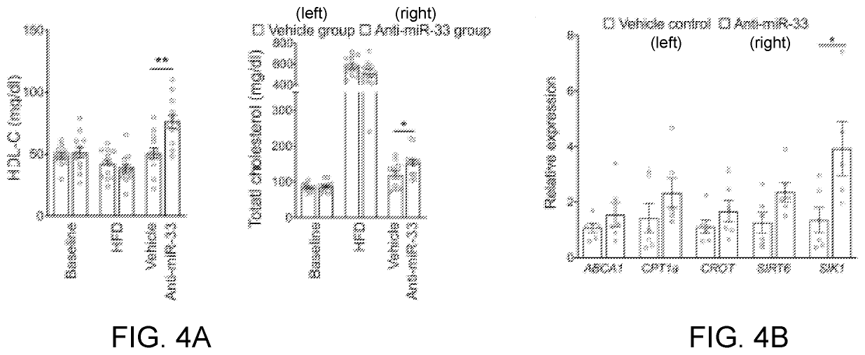

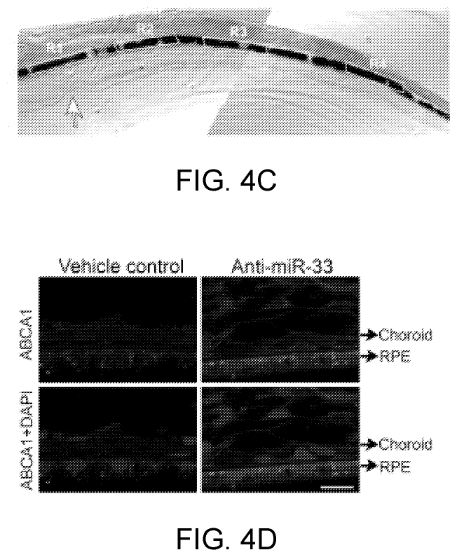

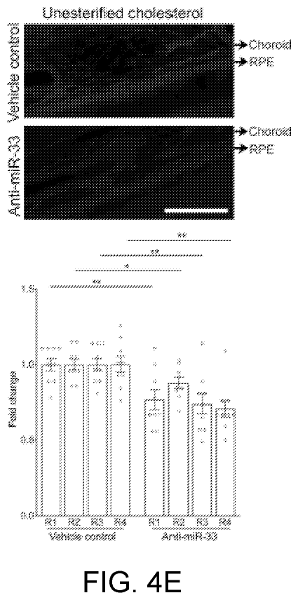

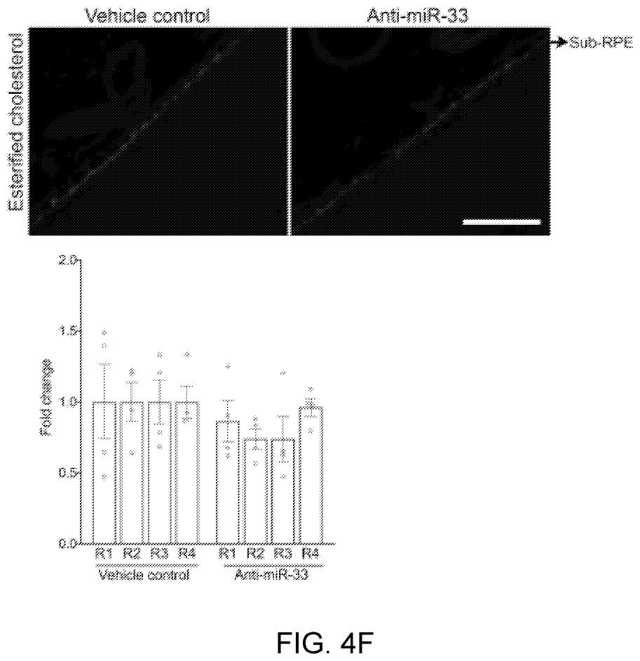

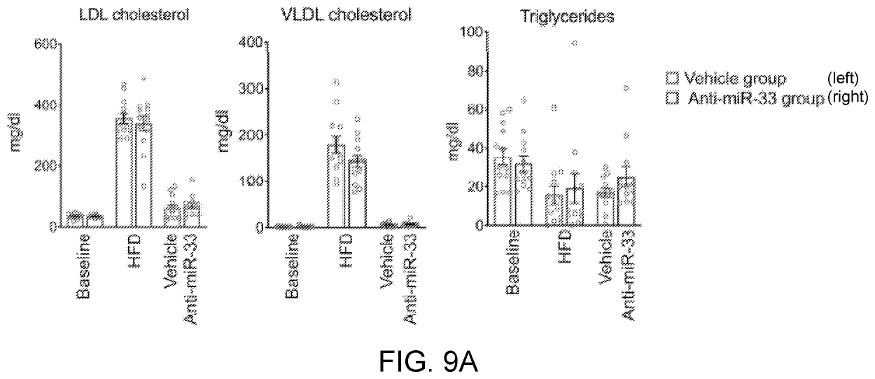

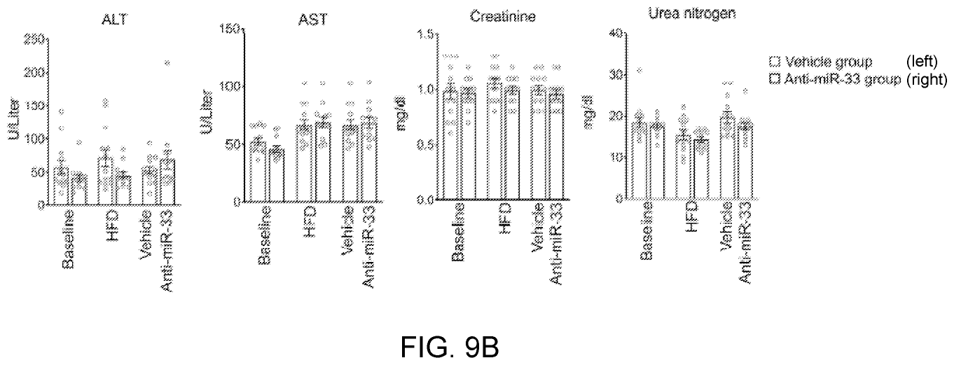

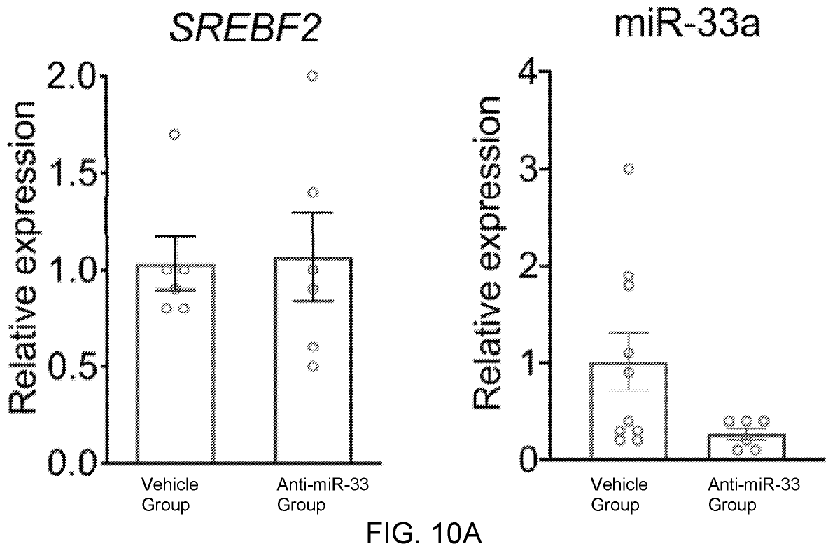

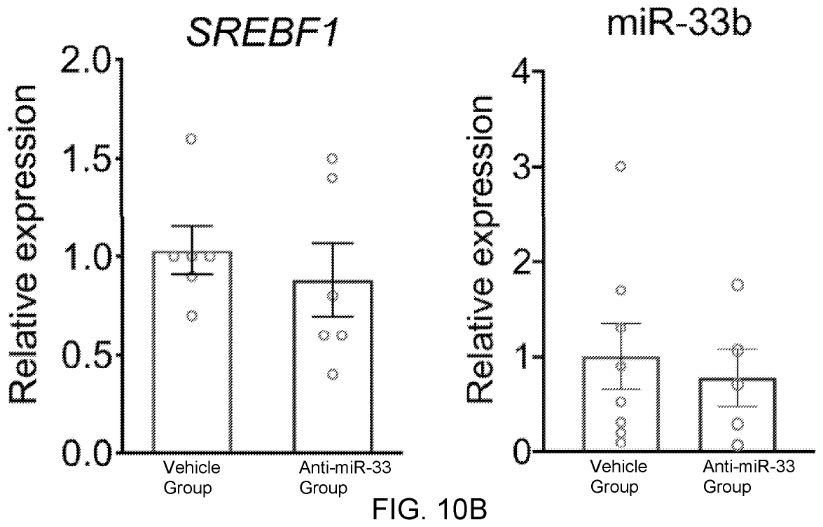

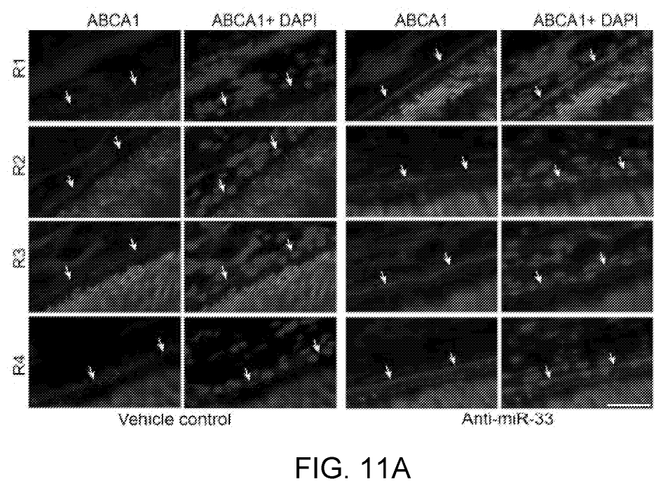

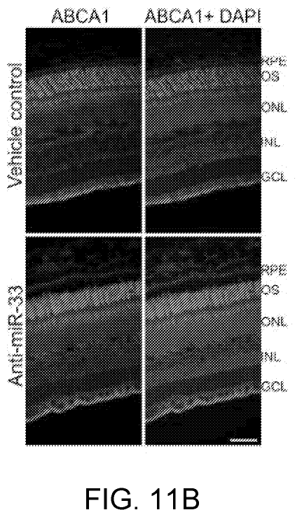

NHPs were fed a WTD diet for 20 months, were then switched to a regular chow diet and concomitantly treated with anti-miR-33 ASO or vehicle control for six weeks. Plasma lipid profiling showed that total cholesterol and HDL cholesterol levels were significantly increased in anti-miR-33 ASO-injected NHPs as compared to vehicle-treated NHPs (FIG. 4A), as expected (22, 24). There was no significant difference between treatment groups in plasma levels of triglycerides, LDL-C, or VLDL-C, nor in levels of liver enzymes ALT/AST and kidney damage markers creatinine and blood urea nitrogen, indicating that the anti-miR-33 ASO treatment exhibited specific effects and was well tolerated (FIGS. 9A and 9B). The eyes collected from NHPs treated with anti-miR33 ASO or vehicle were then evaluated. In RPE cells of NHPs treated with antimiR-33 ASO, the expression levels of miR-33 target genes (ABCA1, CPT1A, CROT, SIRT6, and SIK1) were increased as compared to vehicle injected NHPs (FIG. 4B). Since cholesterol accumulation in the macula of humans is suggested to play a role in AMD pathology (1, 2), we systematically examined the expression of ABCA1, cholesterol carrier lipoprotein, and cholesterol deposition from the fovea up to the peripheral retina of NHPs treated with vehicle or anti-miR-33 ASO (FIG. 4C). Analysis of retinal cryosections demonstrated that ABCA1 protein was markedly increased in the RPE cell layer of anti-miR-33 ASO-treated NHPs as compared to the vehicle-treated NHP from fovea to the periphery (FIG. 4D, FIG. 10A, FIG. 11A), further supporting a direct effect of the anti-miR-33 ASO in the RPE layers of NHPs. The expression pattern of ABCA1 in the neural retina of vehicle- or anti-miR-33-treated NHP retinal sections was not significantly altered (FIG. 10B, FIG. 11B). The eyes collected from NHPs treated with anti-miR-33 ASO or vehicle were also evaluated for SREBF1. SREBF2, miR-33a, and miR-33b expression. Anti-miR-33 ASO treatment resulted in a trend of decreased miR-33a and miR-33b levels in NHP RPE cells (n=6), without any change in the expression of the host genes SREBF1, and SREBF2 (FIGS. 10A and 10B, FIGS. 11A and 11B). Methods: Expression levels of miR-33a, miR-33b, SREBF1, and SREBF2, were measured by quantitative RT-PCR in RPE cells isolated from NHPs fed a high-fat diet for 20 months and then switched to a regular chow diet and injected with anti-miR-33 ASO or vehicle for six weeks (n=6). MicroRNA expression levels were normalized to RNU48 and mRNA expression levels were normalized to PP/H or HPRT1.

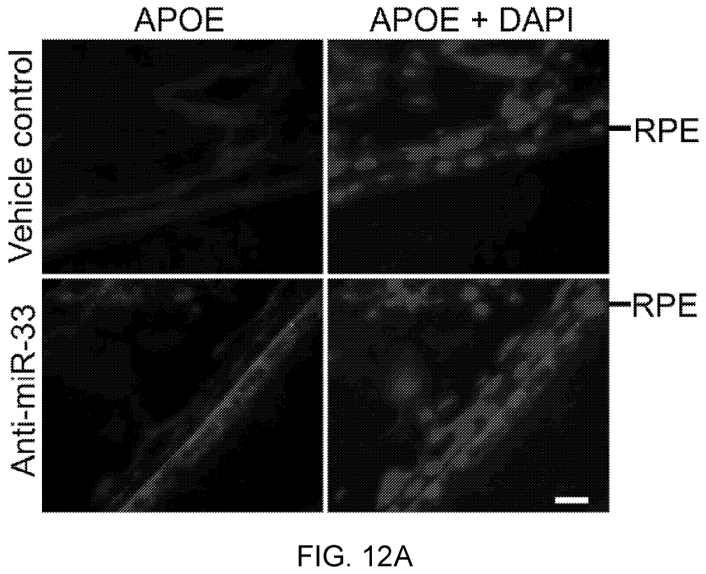

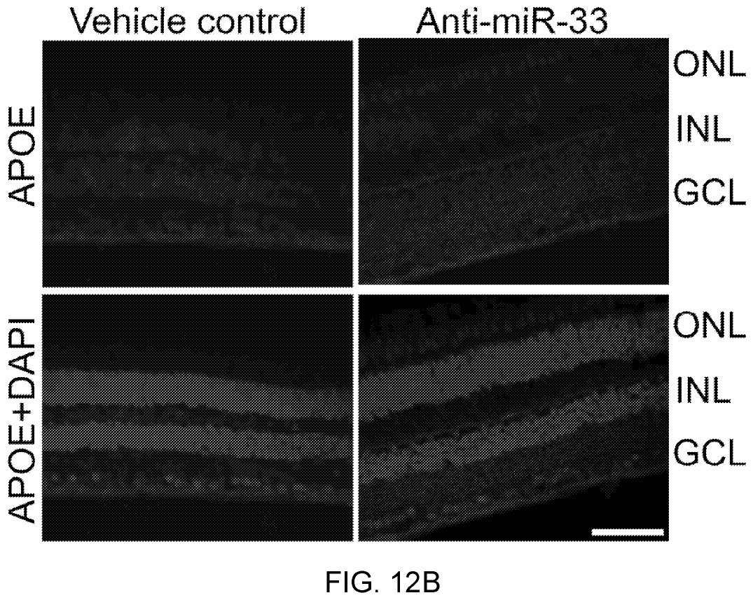

As APOE is also strongly genetically linked to AMD (4, 30) and human RPE-derived APOE regulates directional lipid efflux (31-33), we carried out analysis of APOE levels in the retina of vehicle and anti-miR-33 ASO-treated NHPs. The expression of APOE in the RPE of anti-miR-33 ASO-treated group was greatly increased in comparison to vehicle-treated group (FIG. 12A). We speculate that the effect of anti-miR-33 ASO on APOE expression is indirect via elevated ABCA1 expression (34). As far the APOE staining in the neural retina of anti-miR-33 ASO-treated or vehicle-treated groups, there was no significant change (FIG. 12B). These results reveal beneficial effects of anti-miR-33 ASO treatment on multiple cholesterol regulators genetically linked to AMD in WTD-fed NHPs.

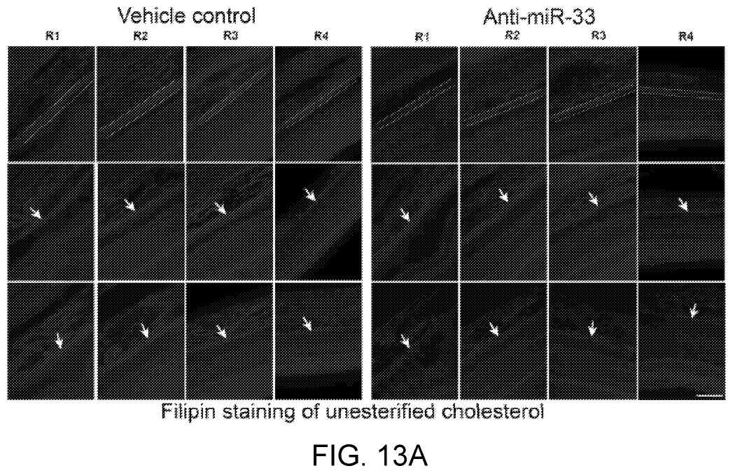

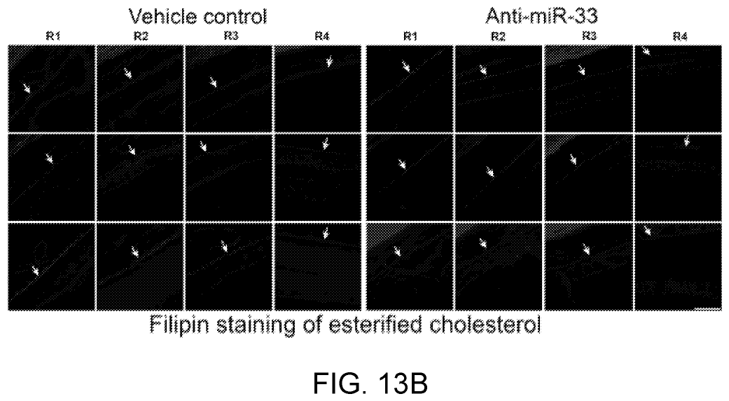

As cholesterol regulators are affected by anti-miR-33 ASO treatment, and the esterified and unesterified forms of cholesterol have been shown to accumulate with age in the macula of human retina (35), we next analyzed NHP retinal sections of vehicle or anti-miR-33 ASO-treated groups with and without cholesterol esterase treatment followed by filipin III staining to label esterified and unesterified cholesterol. The data revealed that compared to the vehicle-treated NHPs, filipin III staining of unesterified cholesterol was significantly reduced in the fovea (P=0.01), parafovea (P=0.01), perifovea (P=0.003), and periphery (P=0.001) of anti-miR-33 ASO-treated NHPs (FIG. 4D, FIG. 13A). With respect to esterified cholesterol, the filipin III staining of esterified cholesterol was mostly detected in the sub-RPE layer from fovea to the central retina (R1-R3) of vehicle or anti-miR-33 ASO-treated NHPs (FIG. 13B), while the staining of esterified cholesterol was very weak in the periphery (FIG. 13B). In the fovea to the central retinal RPE layer, unesterified cholesterol staining was moderately decreased in the antimiR-33 ASO-treated NHP compared to the vehicle treated group (FIG. 4E, FIG. 4F, FIG. 13B). Taken together, these results show that retinal cholesterol levels and key cholesterol trafficking proteins are beneficially impacted upon therapeutic targeting of miR-33a/b in WTD-fed NHPs, in agreement with a pathological role for miR-33a/b in contributing to cholesterol related AMD-like phenotypes in mammals.

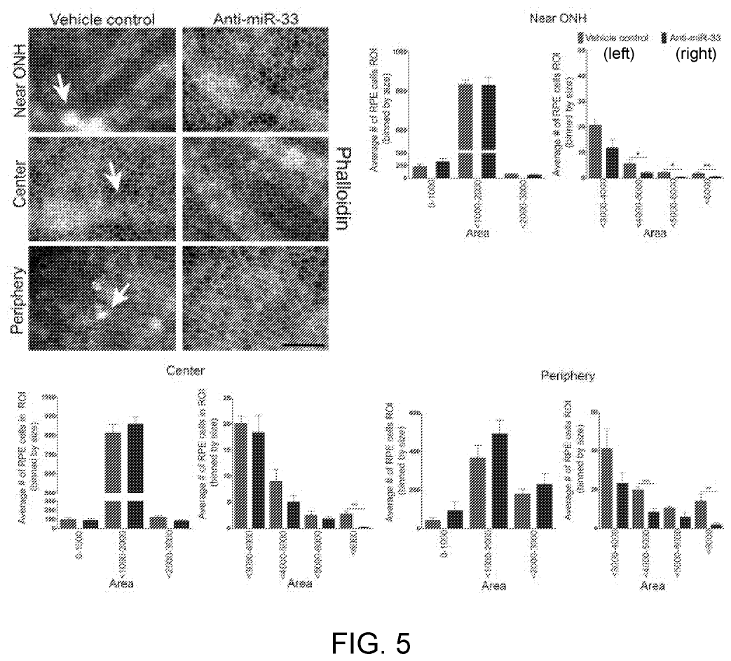

In AMD, RPE cells have been shown to enlarge and undergo morphological changes leading to cell death and atrophy (36). To assess whether miR-33 might contribute to high-fat/cholesterol diet-induced RPE morphological changes, RPE flatmounts were prepared from vehicle- or antimiR-33 ASO-treated NHPs and stained for phalloidin to visualize the actin cytoskeleton and quantify the area of each RPE cells in the regions closer to optic nerve head (ONH), center, and periphery. In comparison to the anti-miR-33 ASO-injected NHPs, vehicle-treated NHPs showed significantly more enlarged RPE cells in all the three regions analyzed (FIG. 5). Particularly in the periphery of vehicle-treated NHPs, the hexagonal RPE shape was severely altered (FIG. 5). These results suggest that miR-33 contributes WTD-induced AMD-like RPE abnormalities in NHPs.

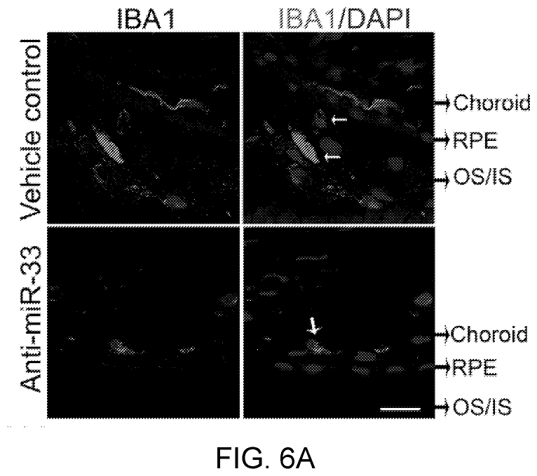

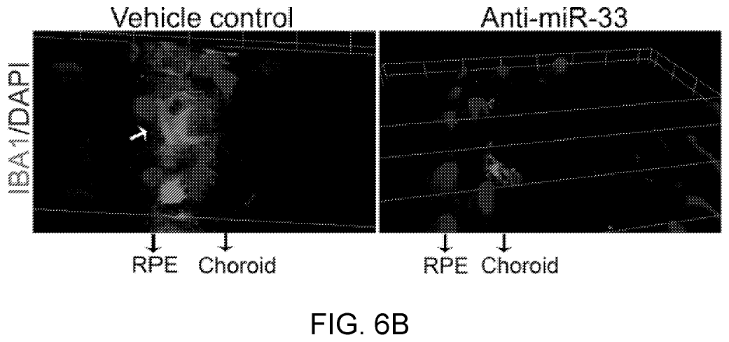

Finally, we assessed retinal inflammation by IBA1 staining (microglia/macrophage marker). Similar to our observations in WTD-fed mice, immune cell infiltration into the RPE-photoreceptor layer and sub-RPE layer was high in vehicle-treated NHP retinal sections in the mid and peripheral region (FIGS. 6A and 6B), as compared to anti-miR-33 ASO-treated NHP retinal sections. This is consistent with a potent pro-inflammatory effect of miR-33 in the retina, in the context of VVTD feeding, akin to what is observed in AMD.

In addition to genetic susceptibility, normal aging and cholesterol deposition with age are postulated to predispose patients to develop AMD (37, 38). Our studies demonstrate that feeding aging mice and non-human primates a high-fat/cholesterol Western-type diet led to cholesterol deposition in the RPE layer, induced RPE morphological and cytoskeletal changes and elicited inflammatory cell recruitment, which are the key clinical features of dry AMD (36, 39). Subcutaneous delivery of anti-miR-33 ASOs reduced cholesterol deposition in the RPE layer, decreased RPE phenotypic changes and suppressed Western-type diet-induced retinal inflammation in aging mice and non-human primates. Macrophages are also thought to be involved in the development and progression of AMD, and cholesterol handling in macrophages is linked to CNV development (wet AMD) (12). Although we cannot definitively conclude whether RPE cells and/or retinal microglia/macrophages were the direct targets of anti-miR-33 ASO treatment, we did find that cholesterol accumulation and inflammation were significantly reduced in the RPE cell layer in response to antimiR-33 ASO treatment.

Even though genetic variants in or near lipid genes and cholesterol/lipid accumulation in the RPE layer are associated with AMD pathogenesis (4, 5, 38), the role for plasma-derived cholesterol/lipids in AMD remains controversial (7, 40, 41). However, accumulating evidence indicates that locally RPE-derived cholesterol and lipoproteins might contribute to cholesterol-rich drusen formation (42-44). Moreover, inflammatory cues elicited by the RPE could promote immune cell infiltration (45, 46). Our results together suggest that miR-33 acts locally in the retina to suppress beneficial RPE cholesterol clearance and stimulate RPE-mediated immune response. The miR-33-dependent cholesterol accumulation and inflammation in the RPE cell layer may thus play a key role in the development of AMD-like pathology, and therapeutic targeting of miR-33 could facilitate the clearance of cholesterol in the RPE cell layer, decrease inflammation and attenuate pathologic changes leading to geographic atrophy, a hallmark of dry AMD.

Methods

Reagents. Precursor miRNAs, including miR-33a, miR-33b, miR-128-1, and miR148a and anti-miRNAs, including miR-33a, anti-miR-33b, anti-miR-128, and antimiR-148a were purchased from Ambion/Thermo Fisher Scientific. Antibodies included: ABCA1 (ab18180, Abcam), SIRT6 (D8D12), vinculin (4650) (Cell Signaling), alpha-tubulin (Calbiochem/EMD Millipore), APOE (NB110-60531), ABCA1 (NB400-105), and Iba1 (NB100-1028) (Novus Biologicals). Other reagents used were: filipin III (Cayman Chemicals), cholesterol esterase (SigmaAldrich), phalloidin-670 (Cytoskeleton, Inc.), cell lysis reagent (Cell Signaling), protein blot blocking buffer (Li-COR Biosciences), TopFluor® cholesterol (Avanti Polar Lipids), APOA1 (Alfa Aesar, LLC) and lipoprotein deficient serum (EMD Millipore). LNA anti-miR and scrambled control oligonucleotides for in vitro and mouse studies were purchased from Exiqon A/S (Vedbaek, Denmark).

Cell culture and transfection. Primary human retinal pigment epithelial (RPE) cells and human RPE cell line (ARPE-19 cells, ATCC) were cultured as described previously (44). Cells were transfected with precursor miRNA (33 nM final concentration), anti-miR (33 nM final concentration) or LNA antisense oligonucleotides (50 nM final concentration) using Lipofectamine RNAiMAX reagent (Life Technologies/Thermo Fisher Scientific). Cell lysates were collected 72 hours post-transfection and 25-30 μg of protein was loaded and separated on SDS-PAGE gel. Transferred protein blots were blocked, incubated with indicated primary and secondary antibodies and examined by Odyssey Imaging System (LiCOR Biosciences). Alpha tubulin or vinculin was used as a loading control for normalization.

Cholesterol efflux assay. ARPE-19 cells were plated at a density of 5×10⁵ per well in a 24-well plate. After attaching for 24 hours, cells were transfected with precursor miRNA or LNA anti-miR, then washed with serum-free DMEM/F-12 (Gibco/Thermo Fisher Scientific) media and incubated with 10 or 25 μM TopFluor® cholesterol for 24 hours. Cells were washed in serum-free media and then incubated with phenol-red free DMEM/F-12 containing 5% lipoprotein deficient serum and 10 μM APOA1 lipoprotein. Supernatant and cell lysates were collected 4 hours post treatment and fluorescence levels were measured using microplate reader (BioTek Instruments Inc.) to calculate the percentage of efflux.

Study of miRNA and gene expression in aging mice. C57BL/6J mice were purchased from Jackson Laboratory, Bar Harbor, Me. and maintained at SERI. Eyes were enucleated at 6, 12, 15, and 18 months. Retinas were dissected out to separate RPE cells, as described previously (47). RPE cells were isolated from six to eight mice per age group. RNA from the RPE pellet was extracted using RNA-Bee (AMS Biotechnology), according to the manufacturer's protocol. Total RNA was then reverse transcribed using iScript™ cDNA synthesis kit (Bio-Rad Laboratories). RT reactions were performed using SYBR Green (Roche) and quantified by real-time PCR (Lightcycler, Roche).

Mouse LNA ASO treatment studies. Twelve-month-old C57BL/6J mice were purchased from Jackson Laboratory and fed a Western-type diet supplemented with 40% kcal from milkfat (Research Diets, INC. D12079B) for four weeks prior to and during treatment. Mice were treated weekly during the four weeks with 10 mg/kg 16-mer LNA anti-miR-33a (5′-ATGCAACTACAATGCA-3′, SEQ ID NO: 1) or scrambled control LNA. LNA ASOs were dissolved in PBS (total volume of 200 μl) then administered subcutaneously. Mice were sacrificed 72 hours after the last injection. Upon sacrifice, ˜1 mL of blood was obtained from mice by right ventricular puncture. Blood was centrifuged at 8,000 rpm for 5 minutes to obtain serum, which was frozen at −80° C. Eyes were enucleated for RNA extraction from RPE cells, cryosectioning, and electron microscopy.

Blood lipid profile and blood chemistry in mice. Total serum cholesterol, triglycerides, aspartate aminotransferase (AST), alanine aminotransferase (ALT), bilirubin, and uric acid levels were determined with a Heska Dri-Chem 4000 Chemistry Analyzer (Heska, Loveland, Colo.) at the MGH Center for Comparative Medicine.

Non-human primate study. Young adult male cynomolgus monkeys (Macaca fascicularis) originated from Mauritius and were an average of 5.0 years of age (range 4.2-6.7) at the onset of the study. The NHPs were housed in an AAALACaccredited facility under the direct care of the University of Kentucky Division of Laboratory Animal Resources. Monkeys were housed in climate-controlled conditions with a 12-hour light and dark cycle. The NHPs were initially ad libitum fed a standard non-human primate diet (Teklad 2050). For the study, the NHPs were singly housed from ˜08:00-15:00 each day and in the morning and afternoon received weighed portions of a semi-synthetic atherogenic diet (see composition in Extended Data Table 1), which provided on average 74 kcal/kg body weight/day. After 20 months on the atherogenic diet, the monkeys were switched back to standard chow diet and were treated for 6 weeks with either vehicle or miR-33a/b antagonist RG428651, a 2′-fluoro/methoxyethyl-modified, phosphorothioate (PS)-backbone-modified, antisense oligonucleotide (5′-TGCAATGCAACTACAATGCAC-3′, SEQ ID NO: 2) (24). Monkeys were injected subcutaneously with vehicle (USP grade saline) or 5 mg ASO/kg body weight twice weekly during the first 2 weeks and then once weekly during the remainder of the study. During the treatment period, animals were singly housed from ˜08:00-15:00 each day and received 12 biscuits of standard diet, which provided on average 64 kcal/kg body weight/day. At the end of the treatment period, the monkeys were fasted overnight and sedated with ketamine (25 mg/kg, IM) and isoflurane (3-5% induction, 1-2% maintenance). After an adequate depth of anesthesia was established by lack of physical response, the inferior vena cava was exposed and cut for exsanguination. A 16-gauge needle was inserted into the left ventricle of the heart and saline was perfused to flush the body of blood. The euthanasia method was deemed acceptable by the American Veterinary Medical Association. After euthanasia, eyes were enucleated for RNA extraction from RPE cell layer and for fixation in 10% formalin for cryosectioning and RPE flatmount preparations. The handling of the NHP eyes was performed at SERI.

Lipid and lipoprotein cholesterol analysis and blood chemistry of non-human primates. After an overnight fast, monkeys were sedated with ketamine (10 mg/kg, IM), body weights were recorded, and blood was collected from the femoral vein into EDTA-containing or serum separation vacutainers. Plasma and serum was isolated by centrifugation at 1,500×g for 30 minutes at 4° C. For determination of circulating concentrations of ALT, AST, creatinine and blood urea nitrogen (BUN), serum was analyzed using the Superchem blood test (ANTECH Diagnostics). Enzymatic assays were used to measure plasma total cholesterol (C7510, Pointe Scientific) and triglycerides (T2449 & F6428, Sigma). The plasma cholesterol distribution among lipoprotein classes was determined after separation by gel filtration chromatography based upon the method described previously (48). An aliquot of plasma was diluted to 0.5 μg total cholesterol/μL in 0.9% NaCl, 0.05% EDTA/NaN₃ and centrifuged at 2,000×g for 10 minutes to remove any particulate debris. The supernatant was transferred to a glass insert contained in a gas chromatography vial, loaded into an autosampler at 4° C. (Agilent Technologies, G1329A), and 40 μL of sample was injected onto a Superose 6 10/300 or Superose 6 Increase 10/300 (GE Healthcare Life Sciences) chromatography column. Under the control of an isocratic pump (Agilent Technologies, G1310A/B), the sample was separated at a flow rate of 0.4 mL/minute with eluent containing 0.9% NaCl, 0.05% EDTA/NaN₃. The column effluent was mixed with total cholesterol enzymatic reagent (C7510, Pointe Scientific), running at a flow rate of 0.125 mL/minute, and the mixture was passed through a knitted reaction coil (Aura Industries Inc., EPOCOD) in a 37° C. H₂O jacket. The absorbance of the reaction mixture was read at 500 nm using a variable wavelength detector (Agilent Technologies, G1314F). The signal was subsequently integrated using Agilent OpenLAB Software Suite (Agilent Technologies). VLDL-C, LDL-C, and HDL-C concentrations were determined by multiplying the TPC concentration by the cholesterol percentage within the elution region for each lipoprotein class.

Quantitative RT-PCR. Total RNA and miRNA were extracted from RPE cells using TriZOL (Life Technologies/Invitrogen) and the mirVana™ miRNA Isolation Kit (Life Technologies/Invitrogen), respectively, according to the manufacturer's instructions. Following extraction, total RNA and miRNA were reverse transcribed using the High Capacity cDNA Reverse Transcription Kit and the TaqMan® MicroRNA Reverse Transcription Kit (Life Technologies/Invitrogen), respectively. RT products were quantified by real time qPCR (Lightcycler, Roche) using the TaqMan® Universal PCR Master Mix. The amount of the indicated mRNA or miRNA was normalized to the amount of B2M mRNA and U6 RNA or snoRNA234 (for mice) or RNU48 (for non-human primates), respectively.

Cryosectioning. Following dissection of the anterior chamber from non-human primates eyes, the eyecup was dissected into four quadrants and the quadrant containing the fovea was cryopreserved by serial treatment with 10, 20, and 30% sucrose solution. Similarly, anterior chamber was dissected from the mouse eyes that were fixed overnight in 4% paraformaldehyde and the posterior eyecup was cryopreserved by serial sucrose solution treatment. The cryopreserved eyecups were embedded in Tissue-Tek® O.C.T compound (SAKURA FINETEK Inc.), frozen and stored at −80° C. Thick retinal frozen sections (12 μm) were cut using a Leica CM3050 S Cryostat. For proper comparison and consistency, retinal sections containing the fovea in all the non-human primates were used for staining. In mice, retinal sections from the optic nerve head regions of all the treatment groups were used for staining.

Filipin III staining of unesterified and esterified cholesterol. Retinal sections were washed in PBS and incubated with filipin III as recommended by the manufacturer for 2 hours at room temperature (RT) to stain unesterified cholesterol. After washing, slides were mounted with ProLong® Gold antifade media (Invitrogen/Thermo Fisher Scientific) and imaged using fluorescent microscope (Nikon Corp.). To stain the esterified cholesterol, retinal sections were incubated in 70% ethanol followed by incubation with cholesterol esterase (1.65 Units/mL) for 2 hours at 37° C. After the enzyme treatment, retinal sections were stained with filipin III and imaged as described above.

Immunofluorescence staining. Retinal cryosections were washed in 1×PBS, blocked in PBS containing 10% goat or donkey serum and 0.05% Triton-X 100 for 1 hour at room temperature. After blocking, sections were incubated with indicated primary and secondary antibodies prepared in PBS containing 2% goat or donkey serum and 0.01% Triton-X 100. After washing, sections were mounted with ProLong® Gold antifade media with DAPI (Invitrogen/Thermo Fisher Scientific) and imaged using fluorescent microscope (Axioscope, Carl Zeiss).

Electron microscopy. Mouse eyes were enucleated and the posterior eyecup was fixed with half strength Karnovsky's fixative (2% formaldehyde+2.5% glutaraldehyde, in 0.1 M sodium cacodylate buffer, pH 7.4; Electron Microscopy Sciences, Hatfield, Pa.) overnight at 4° C. After fixation, mouse eye samples were rinsed with 0.1 M sodium cacodylate buffer, post-fixed with 2% osmium tetroxide in 0.1 M sodium cacodylate buffer for 1.5 hours, en bloc stained with 2% gadolinium (III) acetate hydrate in 0.05 M sodium maleate buffer, then dehydrated with graded ethyl alcohol solutions, transitioned with propylene oxide and resin infiltrated in tEPON-812 epoxy resin (Tousimis, Rockville, Md.), utilizing an automated EMS Lynx 2 EM tissue processor (Electron Microscopy Sciences, Hatfield, Pa.). The processed samples were oriented into tEPON-812 epoxy resin inside flat molds and polymerized in a 60° C. oven. Semi-thin sections were cut at 1 μm thickness then stained with 1% toluidine blue in 1% sodium tetraborate aqueous solution for assessment by light microscopy. Ultrathin sections (80 nm) were cut from each sample block using a Leica EM UC7 ultramicrotome (Leica Microsystems, Buffalo Grove, Ill., USA) and a diamond knife, then collected using a loop tool onto either 2×1 mm, single slot formvar-carbon coated or 200 mesh uncoated copper grids and air-dried. The thin sections on grids were stained with aqueous 2.5% aqueous gadolinium (III) acetate hydrate and Sato's lead citrate stains using a modified Hiraoka grid staining system. Grids were imaged using a FEI Tecnai G2 Spirit transmission electron microscope (FEI, Hillsboro, Oreg.) at 80 kV interfaced with an AMT XR41 digital CCD camera (Advanced Microscopy Techniques, Woburn, Mass.) for digital TIFF file image acquisition. TEM imaging of retina samples were assessed and digital images captured at 2,000×2,000 pixel, 16 bit resolution.

Non-human primate flatmount preparation and analysis. For consistency, retina was gently detached from the quadrant opposite to the fovea and the RPEchoroid layer was carefully separated from the sclera. The RPE-choroid layer was incubated with phalloidin-670 overnight at 4° C., as recommended by the manufacturer. The samples were then washed with PBS and mounted with ProLong® Gold antifade media (Invitrogen/Thermo Fisher Scientific). The areas closer to the optic nerve head, center and periphery were imaged (five images per region) using fluorescent microscope (Axioscope, Carl Zeiss). The area of each RPE cells was quantified using Matlab as described below and the cells were segregated based on size.

MATLAB image quantification methodology. The phalloidin stained RPE cell size was measured using the Matlab module developed by The Nikon Imaging Center, Harvard Medical School. In brief, the images were annotated using the ‘ImageAnnotationBot’ module (https://www.mathworks.com/matlabcentral/fileexchange/64719imageannotationbot). After annotation, the following parameters were set to measure the area of each cell per image, (i) BoundariesThreshold-to obtain binary images, (ii) MinAreaBoundaryComps- to eliminate small components from thresholded images, (iii) DistTransfThreshold- to select markers from distance transform images, (iv) RemoveBoundaryCells- to remove cells in the boundary, (v) SolidityRange- to select nearly convex cells, and (vi) ExtentRange- to create area of shape. MATLAB machine learning module used for cell size quantification will be available at https://hms-idac.github.io/MatBots/.

Statistics. All in vitro experiments were repeated at least three times. A majority of the in vivo data analyses were conducted in a masked manner (except Iba1 staining in non-human primates). Based on a preliminary study we used 10 mice for the LNA ASO study per treatment condition. Four mice from each treatment group were used for histology and the remaining six were used for gene expression studies. None of the mice were excluded from the analysis. All nonhuman primate samples received were analyzed. There were 12 vehicle controls and 12 anti-miR-33 ASO samples for histological studies and nine vehicle controls and six anti-miR-33 ASO samples for gene expression-related studies. All statistical analyses were conducted using GraphPad Prism software and the error bars on the histogram represent ±S.E.M. Statistical differences for age-related gene or miRNA expression studies in mice were analyzed by one-way analysis of variance followed by a post Dunnett's multiple comparisons. Statistical differences for all the other studies were measured using unpaired two-sided Student's t test. P 0.05 was considered as statistically significant.

### REFERENCES CITED IN THE ABOVE EXAMPLES SECTION

1. Xu Q, Cao S, Rajapakse S, Matsubara J A. Understanding AMD by analogy: systematic review of lipid-related common pathogenic mechanisms in AMD, AD, AS and GN. Lipids Health Dis 2018; 17(1):3.
2. Curcio C A, Johnson M, Rudolf M, Huang J D. The oil spill in ageing Bruch membrane. British Journal of Ophthalmology 2011; 95(12):1638-1645.
3. Curcio C A, Johnson M, Huang J D, Rudolf M. Apolipoprotein B-containing lipoproteins in retinal aging and age-related macular degeneration. The Journal of Lipid Research 2010; 51(3):451-467.
4. McKay G J et al. Evidence of association of APOE with age-related macular degeneration: a pooled analysis of 15 studies. Hum. Mutat. 2011; 32(12):1407-1416.
5. Yu Y et al. Association of variants in the LIPCand ABCA1genes with intermediate and large drusen and advanced age-related macular degeneration. Invest. Ophthalmol. Vis. Sci. 2011; 52(7):4663.
6. Yu Y et al. Common variants near FRK/COL10A1 and VEGFA are associated with advanced age-related macular degeneration. Human Molecular Genetics 2011; 20(18):3699-3709.
7. Pennington K L, DeAngelis M M. Epidemiology of age-related macular degeneration (AMD): associations with cardiovascular disease phenotypes and lipid factors. Eye Vis (Lond) 2016; 3:34.
8. Malek G et al. Apolipoprotein E allele-dependent pathogenesis: a model for age-related retinal degeneration. Proc. Natl. Acad. Sci. U.S.A. 2005; 102(33):11900-11905.
9. Bretillon L et al. ApoB100,LDLR−/− Mice exhibit reduced electroretinographic response and cholesteryl esters deposits in the retina. Invest. Ophthalmol. Vis. Sci. 2008; 49(4):1307-1314.
10. Vawas D G et al. Regression of some high-risk features of age-related macular degeneration (AMD) in patients receiving intensive statin treatment. EBIOM 2016; 5(C):198-203.
11. Storti F et al. Impaired ABCA1/ABCG1-mediated lipid efflux in the mouse retinal pigment epithelium (RPE) leads to retinal degeneration. Elife 2019; 8. doi:10.7554/eLife.45100
12. Sene A et al. Impaired Cholesterol Effluxin Senescent Macrophages Promotes Age-Related Macular Degeneration. Cell Metabolism 2013; 17(4):549-561.
13. Ambros V. The functions of animal microRNAs. Nature 2004; 431(7006):350-355.
14. Mendell J T, Olson E N. MicroRNAs in stress signaling and human disease. Cell 2012; 148(6):1172-1187.
15. Rottiers V, Näär A M. MicroRNAs in metabolism and metabolic disorders. Nat Rev Mol Cell Biol 2012; 13(4):239-250.
16. Naar A M et al. Chromatin, TAFs, and a novel multiprotein coactivator are required for synergistic activation by Sp1 and SREBP-1a in vitro. Genes & Development 1998; 12(19):3020-3031.
17. Naar A M et al. Composite co-activator ARC mediates chromatin-directed transcriptional activation. Nature 1999; 398(6730):828-832.
18. Yang F et al. An ARC/Mediator subunit required for SREBP control of cholesterol and lipid homeostasis. Nature 2006; 442(7103):700-704.
19. Walker A K et al. Conserved role of SIRT1 orthologs in fasting-dependent inhibition of the lipid/cholesterol regulator SREBP. Genes & Development 2010; 24(13):1403-1417.
20. Walker A K et al. A conserved SREBP-1/phosphatidylcholine feedback circuit regulates lipogenesis in metazoans. Cell 2011; 147(4):840-852.
21. Najafi-Shoushtari S H et al. MicroRNA-33 and the SREBP host genes cooperate to control cholesterol homeostasis. [Internet]. Science 2010; 328(5985):1566-1569.
22. Rottiers V et al. Pharmacological inhibition of a microRNA family in nonhuman primates by a seed-targeting 8-mer antimiR. Sci Transl Med 2013; 5(212):212ra162.
23. Rayner K J et al. Antagonism of miR-33 in mice promotes reverse cholesterol transport and regression of atherosclerosis. J. Clin. Invest. 2011; 121(7):2921-2931.
24. Rayner K J et al. Inhibition of miR-33a/b in non-human primates raises plasma HDL and lowers VLDL triglycerides. Nature 2011; 478(7369):404-407.
25. Rotllan N, Ramirez C M, Aryal B, Esau C C, Fernandez-Hernando C. Therapeutic silencing of microRNA-33 inhibits the progression of atherosclerosis in Ldlr−/− mice-brief report. Arteriosclerosis, Thrombosis, and Vascular Biology 2013; 33(8):1973-1977.
26. Wagschal A et al. Genome-wide identification of microRNAs regulating cholesterol and triglyceride homeostasis. Nature Medicine 2015; 21(11):1290-1297.
27. Dávalos A et al. miR-33a/b contribute to the regulation of fatty acid metabolism and insulin signaling. Proc. Natl. Acad. Sci. U.S.A. 2011; 108(22):9232-9237.
28. Cao X et al. Macrophage polarization in the maculae of age-related macular degeneration: a pilot study. Pathol. Int. 2011; 61 (9):528-535.
29. Ambati J, Atkinson J P, Gelfand B D. Immunology of age-related macular degeneration. Nature Reviews Immunology 2013; 13(6):438-451.
30. Zareparsi S et al. Association of apolipoprotein E alleles with susceptibility to age-related macular degeneration in a large cohort from a single center. Invest. Ophthalmol. Vis. 2004; 45(5):1306-1310.
31. Ishida B Y. Regulated expression of apolipoprotein E by human retinal pigment epithelial cells. The Journal of Lipid Research 2003; 45(2):263-271.
32. Ishida B Y, Duncan K G, Bailey K R, Kane J P, Schwartz D M. High density lipoprotein mediated lipid efflux from retinal pigment epithelial cells in culture. British Journal of Ophthalmology 2006; 90(5):616-620.
33. Storti F et al. Regulated efflux of photoreceptor outer segment-derived cholesterol by human RPE cells. Experimental Eye Research 2017; 165:65-77.
34. Wahrle S E et al. ABCA1 is required for normal central nervous system ApoE levels and for lipidation of astrocyte-secreted apoE. J. Biol. Chem. 2004; 279(39):40987-40993.
35. Curcio C A, Johnson M, Huang J-D, Rudolf M. Aging, age-related macular degeneration, and the response-to-retention of apolipoprotein B-containing lipoproteins. Progress in Retinal and Eye Research 2009; 28(6):393-422.
36. Ach T et al. Lipofuscin re-distribution and loss accompanied by cytoskeletal stress in retinal pigment epithelium of eyes with age-related macular degeneration. Invest. Ophthalmol. Vis. Sci. 2015:1-35.
37. Ehrlich R et al. Age-related macular degeneration and the aging eye. Clin Interv Aging 2008; 3(3):473-482.
38. Ebrahimi K B, Handa J T. Lipids, Lipoproteins, and Age-Related Macular Degeneration. Journal of Lipids 2011; 2011(3):1-14.
39. Levy O et al. Apolipoprotein E promotes subretinal mononuclear phagocyte survival and chronic inflammation in age-related macular degeneration. EMBO Mol Med 2015; 7(2):211-226.
40. Reynolds R, Rosner B, Seddon J M. Serum Lipid Biomarkers and Hepatic Lipase Gene Associations with Age-Related Macular Degeneration. OPHTHA 2010; 117(10):1989-1995.
41. PhD SBM, DSc GDSM. Mendelian randomization implicates high-density lipoprotein cholesterol associated mechanisms in etiology of age-related macular degeneration. OPHTHA 2017; 124(8):1165-1174.
42. Wang L et al. Lipoprotein particles of intraocular origin in human Bruch membrane: an unusual lipid profile. Invest. Ophthalmol. Vis. Sci. 2009; 50(2):870-877.
43. Johnson L V et al. Cell culture model that mimics drusen formation and triggers complement activation associated with age-related macular degeneration. Proc. Natl. Acad. Sci. U.S.A. 2011; 108(45):18277-18282.
44. Lyssenko N N et al. Directional ABCA1-mediated cholesterol efflux and apoBlipoprotein secretion in the retinal pigment epithelium. J. Lipid Res. 2018; 59(10):1927-1939.
45. Gnanaguru G, Choi A R, Amarnani D, D'Amore P A. Oxidized lipoprotein uptake through the CD36 receptor activates the NLRP3 inflammasome in human retinal pigment epithelial cells. Invest. Ophthalmol. Vis. Sci. 2016; 57(11):4704-4712.
46. Liu J et al. Impairing autophagy in retinal pigment epithelium leads to inflammasome activation and enhanced macrophage-mediated angiogenesis. Sci Rep 2016; 6:20639.
47. Wang C X-Z, Zhang K, Aredo B, Lu H, Ufret-Vincenty R L. Novel method for the isolation of RPE cells specifically for RNA extraction and analysis. Experimental Eye Research 2012; 102(C):1-9.
48. Kieft K A, Bocan™, Krause B R. Rapid on-line determination of cholesterol distribution among plasma lipoproteins after high-performance gel filtration chromatography. The Journal of Lipid Research 1991; 32(5):859-866.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the claims.

What is claimed is:

1. An oligonucleotide comprising a total of 7 to 50 interlinked nucleotides and having a nucleobase sequence comprising at least one bridged nucleic acid and at least 6 contiguous nucleobases complementary to an equal-length portion within an miR-33 target nucleic acid.

2. The oligonucleotide of claim 1, wherein the oligonucleotide is an antisense oligonucleotide.

3. The oligonucleotide of claim 1, wherein the oligonucleotide is a single-stranded oligonucleotide.

4. The oligonucleotide of claim 1, wherein the oligonucleotide is a unimer, and wherein each of the nucleotides is independently a bridged nucleic acid.

5. The oligonucleotide of claim 1, wherein the bridged nucleic acid is a locked nucleic acid or ethylene bridged nucleic acid.

6. The oligonucleotide of claim 5, wherein the bridged nucleic acid is a locked nucleic acid.

7. The oligonucleotide of claim 1, wherein the oligonucleotide comprises a total of 7 to 30 nucleotides.

8. The oligonucleotide of claim 1, wherein the oligonucleotide comprises a total of 14 to 23 nucleotides.

9. The oligonucleotide of claim 1, wherein the miR-33 target nucleic acid is pri-miR-33a, pre-miR-33a, or miR-33a.

10. The oligonucleotide of claim 1, wherein the miR-33 target nucleic acid is pri-miR-33b, pre-miR-33b, or miR-33b.

11. The oligonucleotide of claim 1, wherein the nucleobase sequence is 5′-ATGCAACTACAATGCA-3′ (SEQ ID NO: 1).

12. The oligonucleotide of claim 1, wherein the nucleobase sequence is 5′-TGCAATGCAACTACAATGCAC-3′ (SEQ ID NO: 2).

13. A recombinant adeno-associated viral (rAAV) particle comprising a nucleic acid vector that comprises a heterologous nucleic acid region comprising a sequence that encodes an interfering RNA comprising a region complementary to an miR-33 target nucleic acid.

14. The rAAV particle of claim 13, wherein the miR-33 target nucleic acid is pri-miR-33a, pre-miR-33a, or miR-33a.

15. The rAAV particle of claim 13, wherein the miR-33 target nucleic acid is pri-miR-33b, pre-miR-33b, or miR-33b.

16. The rAAV particle of claim 13, wherein the interfering RNA is shRNA or siRNA.

17. The rAAV particle of claim 13, wherein the sequence is operably linked to a promoter.

18. The rAAV particle of claim 17, wherein the promoter is capable of expressing the interfering RNA in a subject's eye.

19. The rAAV particle of claim 17, wherein the promoter is a hybrid chicken β-actin (CBA) promoter or an RNA polymerase III promoter.

20. The rAAV particle of claim 13, wherein the vector comprises AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype inverted terminal repeats.

21. The rAAV particle of claim 13, wherein the particle comprises an AAV1, AAV2, AAV3, AAV4, AAV5, AAV6, AAV7, AAV8, AAVrh8, AAVrh8R, AAV9, AAV10, AAVrh10, AAV11, AAV12, a tyrosine capsid mutant, a heparin binding capsid mutant, an AAV2R471A capsid, an AAVAAV2/2-7m8 capsid, an AAV DJ capsid, AAV2 N587A capsid, AAV2 E548A capsid, AAV2 N708A capsid, AAV V708K capsid, goat AAV capsid, AAV1/AAV2 chimeric capsid, bovine AAV capsid, mouse AAV capsid, or rAAV2/HBoV1 capsid.

22. A pharmaceutical composition comprising a pharmaceutically acceptable excipient and the oligonucleotide of any one of claims 1 to 11 or the rAAV particle of any one of claims 12 to 21.

23. A method of treating age-related macular degeneration in a subject in need thereof, the method comprising administering to the subject a therapeutically effective amount of the oligonucleotide of any one of claims 1 to 11 or the rAAV particle of any one of claims 12 to 21.

24. A method of treating age-related macular degeneration in a subject in need thereof, the method comprising administering to the subject a therapeutically effective amount of an miR-33 inhibitor.

25. The method of claim 24, wherein the miR-33 inhibitor is an antisense oligonucleotide, shRNA, siRNA, or an rAAV particle comprising a nucleic acid vector that comprises a heterologous nucleic acid region comprising a sequence that encodes the miR-33 inhibiting antisense oligonucleotide, shRNA, or siRNA.

26. A method of treating age-related macular degeneration in a subject in need thereof, the method comprising administering to the subject a therapeutically effective amount of:
(i) an oligonucleotide comprising a total of 7 to 50 interlinked nucleotides and having a nucleobase sequence comprising at least 6 contiguous nucleobases complementary to an equal-length portion within an miR-33 target nucleic acid;
or
(ii) a recombinant adeno-associated viral (rAAV) particles comprising a nucleic acid vector that comprises a heterologous nucleic acid region comprising a sequence that encodes the oligonucleotide.

27. The method of claim 26, wherein the method comprises administering a therapeutically effective amount of the oligonucleotide.

28. The method of claim 27, wherein the oligonucleotide is a single-stranded oligonucleotide.

29. The method of claim 27, wherein the oligonucleotide is an antisense oligonucleotide.

30. The method of claim 28, wherein the oligonucleotide comprises at least one modified sugar nucleoside.

31. The method of claim 30, wherein at least 50% of the nucleosides in the oligonucleotide comprise the modified sugar nucleoside.

32. The method of claim 30, wherein all nucleosides in the oligonucleotide comprise the modified sugar nucleoside.

33. The method of claim 30, wherein the modified sugar nucleoside is a 2′-modified sugar nucleoside.

34. The method of claim 33, wherein the 2′-modified sugar nucleoside comprises a 2′-modification independently selected from the group consisting of 2′-fluoro, 2′-methoxy, and 2′-methoxyethoxy.

35. The method of claim 30, wherein the modified sugar nucleoside is a bridged nucleic acid.

36. The method of claim 27, wherein the oligonucleotide is a gapmer comprising a 5′-wing, a 3′-wing, and a gap; wherein each of the 5′-wing and the 3′-wing comprises a total of 1 to 5 nucleotides, each of which is independently a bridged nucleic acid, and each nucleotide in the gap a deoxyribonucleotide.

37. The method of claim 35, wherein the bridged nucleic acid is a locked nucleic acid or ethylene bridged nucleic acid.

38. The method of claim 37, wherein the bridged nucleic acid is a locked nucleic acid.

39. The method of claim 27, wherein at least one internucleoside linkage in the oligonucleotide is a phosphorothioate diester.

40. The method of claim 39, wherein at least 50% of internucleoside linkages in the oligonucleotide are phosphorothioate diesters.

41. The method of claim 40, wherein all internucleoside linkages in the oligonucleotide are phosphorothioate diesters.

42. The method of claim 27, wherein the nucleobase sequence is 5′-ATGCAACTACAATGCA-3′ (SEQ ID NO: 1).

43. The method of claim 27, wherein the nucleobase sequence is 5′-TGCAATGCAACTACAATGCAC-3′ (SEQ ID NO: 2).

44. The method of claim 28, wherein the oligonucleotide comprises a total of 7 to 30 nucleotides.

45. The method of claim 28, wherein the oligonucleotide comprises a total of 14 to 23 nucleotides.

46. The method of claim 27, wherein the method comprises administering the oligonucleotide as a guide strand in an siRNA.

47. The method of claim 26, wherein the method comprises administering the rAAV particle.

48. The method of claim 47, wherein the rAAV particle is that of any one of claims 13 to 21.

49. The method of claim 26, wherein the miR-33 target nucleic acid is pri-miR-33a, pre-miR-33a, or miR-33a.

50. The method of claim 26, wherein the miR-33 target nucleic acid is pri-miR-33b, pre-miR-33b, or miR-33b.

51. The method of claim 26, wherein the route of administration is an intraocular injection, intravitreal injection, subretinal injection, topical application, implantation, intraperitoneal injection, intramuscular injection, subcutaneous injection, or intravenous injection.
